(19) United States
(12) Patent Application Publication
(10) Pub. No.: US 2006/0183119 A1
(43) Pub. Date: Aug. 17, 2006

(54) Method for determining predisposition to a physiological reaction in a patient

(75) Inventor: Chantal Guillemette, Quebec (CA)

Correspondence Address: OGILVY RENAULT LLP, 1981 MCGILL COLLEGE AVENUE, SUITE 1600, MONTREAL, QC, H3A2Y3, CA

(73) Assignee: UNIVERSITE LAVAL, Quebec (CA)

(21) Appl. No.: 10/528,463

(22) PCT Filed: Aug. 20, 2003
(86) PCT No.: PCT/CA03/01269

§ 371 Date: Mar. 21, 2005

Related U.S. Application Data
(60) Provisional application No. 60/412,002, filed on Sep. 20, 2002.

(51) Int. Cl. C12Q 1/68

(52) U.S. Cl. 435/6

(57) ABSTRACT

The present invention relates to a method for determining predisposition to a physiological reaction in a patient. Particularly, the present invention relates to a method for determining a predisposition to toxicity induced by a camptothecin analog or to an immunosuppressive mycophenolic acid-based therapy. This method comprises the characterization of nucleic acid sequences from the patient. The nucleic acid sequence encodes for an amino acid sequence or regulates the expression of UGT1A1, UGT1A7, UGT1A9 or their polymorphic variants. The method also comprises the analysis of haplotypic variation within these genes.

### TECHNICAL FIELD

The present invention relates to a method for determining predisposition to a physiological reaction to a xenobiotic, a drug or an endogenously secreted compound, in a patient. Particularly, the present invention consists in a method comprising the characterization of a nucleic acid sequence from a patient. These nucleic acid sequences encode for amino acid sequences or regulate the expression of genes.

### BACKGROUND ART

Recent evidences support the concept that polymorphic variation in genes encoding metabolism enzymes (MEs) are likely to play an important role in clinical response to therapeutic drugs and in exogenous or endogenous compound elimination.

Interindividual variations in response to a drug or to exogenous or endogenous compounds can be classified in three groups. The first segment of the population is known as poor metabolizers (PMs). These individuals often show accumulation of drugs or metabolites caused by a genetic defect in metabolizing enzymes and increased predisposition to adverse drug reactions is an important consequence of PM genotypes. In opposite, ultrarapid metabolizers (UMs) eliminate drugs excessively rapidly from the body. These patients, for example, do not develop sufficient high plasma levels of drugs and therefore do not respond to treatments, also giving rise to both clinical and economical complications. The remaining proportion of the population categorized as “normal” patients are named extensive metabolizers (EMs).

Some researchers have studied pharmacogenetics of human drug-metabolizing enzymes (DME), more specifically enzymes of the glucuronidation pathway and have demonstrated that glucuronidation, like other DME pathways, is also subject to interindividual variations. The glucuronidation reaction is catalyzed by UDP-glucuronosyltransferase enzymes (UGTs), a set of enzymes that increase the polarity of xenobiotics, drugs and endogenous compounds to facilitate their excretion from the body. Glucuronidation reaction occurs on different functional groups that Include hydroxyl, carboxyl, amino and sulfur. UGTs have the most important effect in both detoxification and promotion of excretion, via both urine and bile. Apart from being a major biochemical pathway well known for its role in drug metabolism, the glucuronidation system is also clearly involved in the homeostasis of numerous endogenous molecules, including steroids, thyroid hormones and bile acids.

Any perturbation in the glucuronidation pathway has the potential to modify the elimination, the detoxification or the pharmacokinetic parameters of a given drug, and consequently drug clearance. As a result, in situations where the activity of the glucuronidation pathway is reduced, it is to be expected that changes in the biological activity, sometimes toxicity, of the compounds will ensue. Therefore, the human genetic variations leading to differences in the is glucuronidation rates could influence the activity of drugs and other chemicals, which undergo this conjugation.

As example, SN-38 or 7-ethyl-10-hydroxycamptothecin, which is the pharmacologically active metabolite of the anticancer drug irinotecan, undergoes extensive glucuronidation in human to form SN-38-G (10-O-glucuronyl-SN-38) and goes through significant binary excretion and enterohepatic circulation. This drug is used globally in the first line treatment of advanced metastatic colorectal cancer (CRC). A major drawback of irinotecan-based chemotherapy is the high incidence of severe hematological and gastrointestinal toxicities, such as diarrhea. Diarrhea is believed to be secondary to the binary excretion of SN-38, the extent of which is determined by SN-38 glucuronidation. Incidences of irinotecan-induced diarrhea can be serious and do not respond adequately to conventional antidiarrheal agents. It is believed that SN-38-G can be deconjugated to form SN-38 by intestinal glucuronidase enzyme, and further causes diarrhea by direct enteric injury. An inverse relationship between SN-38 glucuronidation rates and severity of diarrhea incidences in patents treated with irinotecan has been shown. These findings indicated that glucuronidation of SN-38 protects against irinotecan-induced gastrointestinal toxicities. Therefore, the conversion of SN-38 to SN-38-G by both hepatic and intestinal UGTs is a critical step in the sequential metabolic pathway of irinotecan, and consequently in drug response and toxicity. Over the existing human UGTs, UGT1A1, UGT1A7 and UGT1A9 are known in the art to be SN-38 conjugators. On the other hand, UGT1A1 and UGT1A9 are highly expressed in the liver, the primary organ involved in the detoxification of irinotecan, and also in the gastrointestinal tract (GI) where toxicity takes place.

Mycophenolic acid (MPA) is also an extensively glucuronidated drug for which an interindividual variation of glucronidation rates is observed. MPA is a metabolite of mycophenolate mofetil (MMF), and is commonly used as immunosuppressive agent. As MPA is known to be conjugated exclusively by the liver UGT1A9, interindividual variation observed with this substrate is therefore attributable only to the UGT1A9 isoform. The study of UGT1A9 is polymorphic variations thus plays a critical role in the control of immunosuppressive therapies and management of graft rejection.

Genetic variations among UGT isoforms have been demonstrated to be also implicated in the Interindividual physiological response to drug administration. Therefore, glucuronidation pathway represented a target for many groups as a way to control irinotecan-associated side effects.

As example, international patent publication number WO 96/01127 describes a method and pharmaceutical compositions to reduce side effects of camptothecin analogs such as irinotecan, therefore reducing associated side effects. This reduction of toxicity would occur by reducing biliary transport or increasing UGT activity, by administrating concomitantly a transport inhibitor or an UGT inducer.

U.S. Pat. No. 6,395,481 reports a method for detecting TA repeats polymorphic variations within the promoter region of the UGT1A1 gene to evaluate predispositions to drug sensitivity associated with low levels of UGT enzymes expression.

International patent publication number WO 02/48400 reports a method for estimating the susceptibility In an individual to adverse side effects caused by the administration of irinotecan. This method Is also based on the evaluation of the TA repeats within the promoter region of UGT1A1, but also includes the analysis of single nucleotide polymorphisms at two other positions within the exon 1.

International patent publication number WO 03/013536 reports a method for selecting a suitable iriontecan therapy for a cancer patient that comprises determining whether the patient has one or multiple variant alleles of the UGT1A1 gene and adjusting irinotecan dosage and/or UGT1A1-modulating drugs consequently.

Considering overlapping substrate specificities of UGT enzymes, it is noteworthy that a higher expression of UGT1A1 protein resulting from an increased gene expression could complement a deficient glucuronidation activity of an altered UGT1A9 protein, or the contrary. An individual harboring two mutated genotypes would therefore have a normal phenotype and is less susceptible to develop a toxicity to a drug than a patient having the low metabolizer phenotype. Therefore, the genotyping studies that consider only one gene encoding a xenobiotic conjugating enzyme are less likely to be accurate than a global analysis of the whole set of genes.

Based on the state of the prior art described hereinabove, it would be highly desirable to be provided with a new diagnostic tool to determine accurately a predisposition to physiological adverse response following drug administration in standard conditions. This would allow to provide physicians with guiding means in determining drugs to be used in a specific treatment.

### DISCLOSURE OF INVENTION

One aim of the present invention is to provide a method for determining a predisposition to a physiological reaction of an individual to a biologically active compound. This method comprises characterizing nucleotide sequence of the individual for at least one of the UGT1A1, UGT1A7 or UGT1A9 gene, or a pert thereof. The presence of at least one polymorphic or haplotypic variation in this nucleotide sequence is indicative of the predisposition to the physiological reaction.

In accordance with the method described herein, the predisposition may be a s hereditary predisposition and the physiological reaction in the patient may be a beneficial reaction, an adverse reaction or a side effect to a compound.

Another aim of the present invention Is to provide a method wherein determining the genetic sequence comprises determining the presence of at least one polymorphic or haplotypic variation in UGT1A1, UGT1A7 or UGT1A9 gene. These variations may include variations of the number of TA repeats in a TATA box of the UGT1A1 gene, C⁻²²⁰⁸T substitution, C⁻²¹⁵²T substitution, C⁻²¹⁴¹T substitution, T¹⁸⁸⁷G substitution, T¹⁸¹⁸C substitution, C⁻⁶⁶⁵T substitution, T⁴⁴⁰C substitution, C⁻³³¹T substitution, T²⁷⁵A substitution, G⁻⁸⁷A substitution, G⁸A missence mutation, a T⁹⁸C missence mutation, or a combination of these variations In the UGT1A9 gene. Alternatively and/or additionally, G³⁵³T, T³⁹⁷G, C⁴⁰¹A, G⁴⁰²A, G⁴²⁷C or T⁶³²C missense mutations can be determined in the UGT1A7 gene.

Another aim of the present invention is to provide a nucleotide sequence for determining a predisposition to a physiological reaction comprising at least one nucleotide sequence selected from the group consisting of SEQ ID NO: 36 to SEQ ID NO: 68, or the complementary sequences thereof.

For the purpose of the present invention the following terms are defined below.

The expression “adverse physiological reaction” is intended to mean any physiological reaction that provides a negative physiological effect to an individual.

The term “ASO” is intended to mean Allele Specific Oligonucleotide analysis.

The term “ASP” is intended to mean Allele Specific PCR analysis.

The expressions “beneficial physiological reaction” or “beneficial reaction” are intended to mean any physiological reaction that provides a positive physiological effect to an individual.

The term “BPD” is intended to mean benzo(a)pyrene-trans-7,8-dihydrodiol.

The term “CPT-11” is intended to mean 7-ethyl-10-[4-(1-piperidino)-1-piperidino]carbonyloxy camptothecin.

The term “DHPLC” is intended to mean denaturing high-performance liquid chromatography.

The term “gene” is intended to mean a segment of nucleic acid involved in producing a polypeptide chain; it includes regions preceding the coding region (promoter, leader sequence), regions following coding region (trailer) and intervening sequences (introns) between individual coding segments (exons).

The term “GI” is intended to mean gastrointestinal tract.

The term “MPA” us intended to mean mycophenolic acid.

The term “PhlP” is intended to mean 2-amino-1-methyl-6-phenylimidazo[4,5-b]pyridine.

The term “RFLP” is intended to mean Restriction Fragment Length Polymorphism analysis.

The term “SN-38” is intended to mean 7-ethyl-10-hydroxycamptothecin.

The term “SSCP” is intended to mean Single Strand Conformation Polymorphism analysis.

The term “UGT” is intended to mean uridine diphospho-glucuronosyltransferase.

### BRIEF DESCRIPTION OF THE DRAWINGS

FIG. 1 illustrates the metabolic pathway of irinotecan hydroclorine (CPT-11);

FIG. 2 illustrates the entero-hepatic cycle of irinotecan biotransformation;

FIG. 3 Illustrates the major role of UGT1A9 in SN-38 glucuronidation;

FIG. 4 illustrates the distribution of SN-38-G formation by human liver samples.

FIGS. 5a to 5f illustrate methods for detecting SNPs;

FIGS. 6a to 6d illustrate the missence mutations in the human first exons of UGT1A7 and UGT1A9 genes;

FIG. 7 Illustrates the expression of the UGT1A9 and UGT1A9 proteins in human liver microsomes;

FIGS. 8a to 8c illustrate the effect of UGT1A9 promoter polymorphisms on protein expression;

FIG. 9 illustrates the effect of the UGT1A9 (−2152) polymorphic variation on MPA glucuronidation activity;

FIG. 10 illustrates the effect of the UGT1A9 (−1818) polymorphic variation on SN-38 glucuronidation activity;

FIGS. 11a to 11d Illustrate the effect of the UGT1A9 (−665) polymorphic variation on glucuronidation activity;

FIG. 12 illustrates the effect of UGT1A9 (−275) polymorphic variation on MPA glucuronidation activity;

FIGS. 13a and 13b illustrate the correlation between the UGT1A9 protein expression and glucuronidation activity;

FIGS. 14a to 14d illustrate the relative expression of UGT1A7 and UGT1A9 protein and their relative activities on SN-38;

FIGS. 15a to 15c Illustrate the glucuronidation rates of the variant UGT1A9 allozymes;

FIGS. 16a to 16l illustrate the immunofluorescence localization of UGT1A9*1, UGT1A9*2 and UGT1A9*3;

FIGS. 17a to 17c illustrate the relationship between UGT1A1 TATA box polymorphic variations and protein expression or glucuronidation activity;

FIGS. 18a and 18b illustrate the correlative association between UGT1A1 protein expression and glucuronidation activity; and

FIG. 19 illustrates the predictive value of the haplotype determination of UGT1A9 and UGT1A1; and

FIGS. 20a and 20b illustrate a sequence alignment of UGT1A proteins at selected positions.

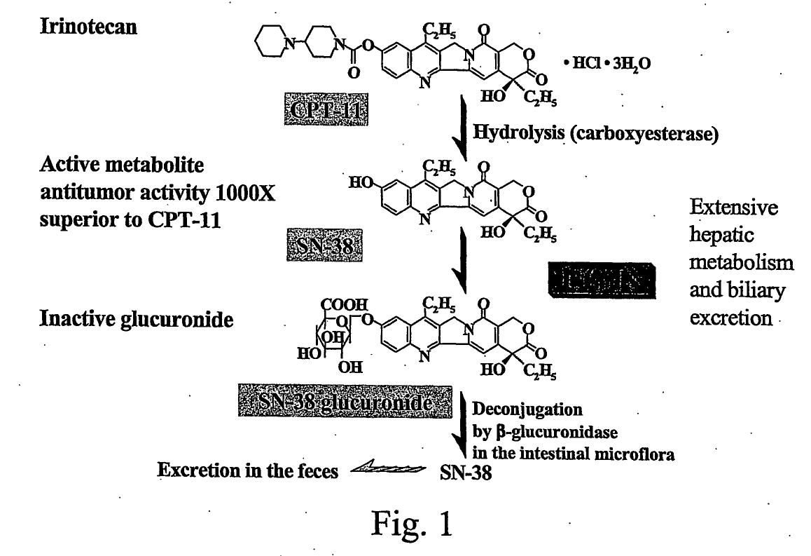

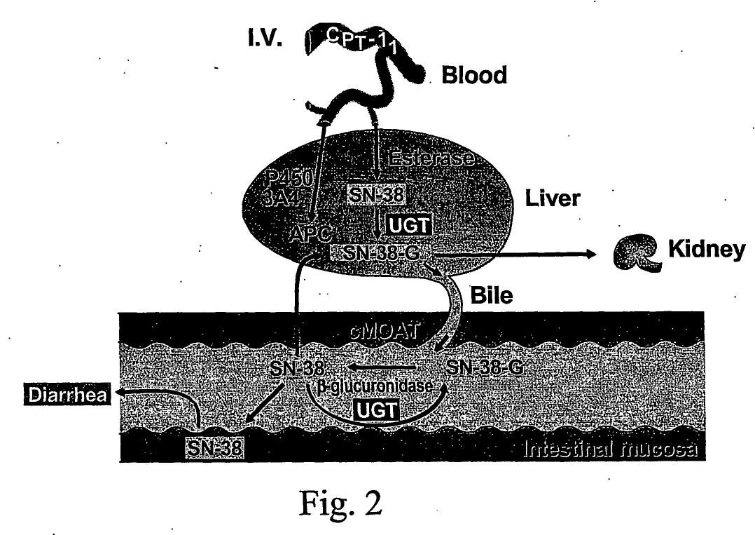

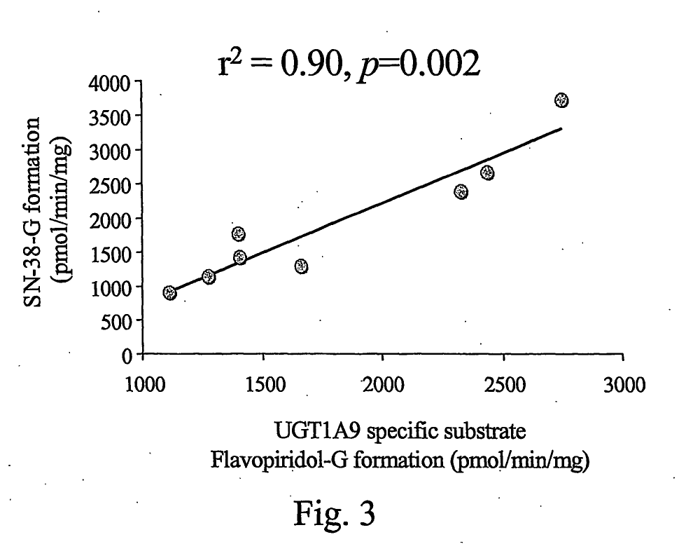

### MODES OF CARRYING OUT THE INVENTION

In accordance with the present invention, there Is provided a method for determining a predisposition to a physiological reaction in an individual comprising characterizing nucleotide sequence of at least one of the UGT1A1, is UGT1A7 or UGT1A9 gene or a part thereof of the individual, where the nucleotide sequence is indicative of the predisposition to a physiological reaction. The individual of the present Invention is a human or an animal, but is preferably a patient having a colorectal cancer or a solid tumor. The predisposition determined with the present method is any higher or lower susceptibility, sensibility, diathesis, proneness, proclivity, tendency, sensitivity, responsiveness, resistance or constitutional sickness to the physiological reaction. This predisposition may be a hereditary predisposition, a non-hereditary congenital predisposition or an acquired predisposition.

The physiological reaction of the present invention comprises a beneficial reaction to a compound, an adverse reaction to a compound or a side effect. Among predisposition to an adverse physiological reaction to a compound, toxicity induced by an anti-cancer drug or a decreased responsiveness to an immunosuppressive agent are preferred. Toxicity to drug may be caused by an increased concentration of the drug in plasma, this increased concentration being attributable to a lower glucuronidation metabolism of this compound or a decreased responsiveness to a drug, the latter being induced by an excessive glucuronidation-mediated elimination form of this compound from the organism. An anti-cancer agent that can be targeted through carrying out the present Invention can be a camptothecin analog, such as 7-ethyl-10-[4-(1-piperidino)-1-piperidino]carbonyloxy camptothecin (irinotecan, CPT-11 ) or 7-ethyl-10-hydroxycamptothecin (SN-38).

As CPT-11 or its active metabolite SN-38 are topoisomerase inhibitors, cells showing higher levels of these enzymes are likely more sensitive to topoisomerase inhibition. Resistance to the drug occurs generally in cells that have low levels of topoisomerase. Resistance to irinotecan may also result from reduced conversion of the inactive prodrug CPT-11 to SN-38, attributable to reduced enzyme levels or, possibly, enzyme mutations. Additionally, an increased catabolic processing of the inhibitors contributes to reduce their is availability within the cell, lowers inhibitor activity and favors drug resistance. It has also been reported that human colon tumors express high levels of the multiple-drug-resistance (MDR1) proteins. This class of enzyme may limit access of certain drugs to cells. In vitro data have demonstrated that camptothecin and its noncharged derivatives such as irinotecan overcome MDR1-mediated resistance. MDR1-mediated resistance to irinotecan may result from its rapid passive diffusion, its absence of interaction with MDR1, or a combination of both characteristics.

Alternatively, the sensitivity to drugs, as for example anticancer drugs, can be observed in cell lines deficient in DNA repair mechanisms. Indeed, DNA repair mechanisms can reverse drug-induced damage caused to the DNA. Therefore, DNA damage that goes unrepaired may result in significant genetic alterations or apoptosis.

The adverse physiological reaction as intended herein does not include the side effects observed with the majority of the population treated with the drug, but comprises physiological reactions that cause more serious threats in particular patients than what is generally expected with that drug In a majority of patients.

In fact, the susceptibility, sensitivity, responsiveness or resistance is higher or lower to what is observed in a patient having an anticipated physiological reaction to the same drug. These adverse physiological reactions are generally traduced by gastrointestinal, hematologic, hepatic, dermatologic, respiratory and neurologic disorders. Although gastrointestinal adverse reactions include nausea and vomiting, the most preoccupying and severe side effect observed is diarrhea. It has been observed that this particular toxicity is attributable to an accumulation of unconjugated SN-38 in the intestine. As SN-38 metabolization rates inversely correlate with the intensity of diarrhea in patients treated with increasing doses of CPT-11, the interindividual differences in pharmacokinetics of SN-38 are suggested to be responsible for the variation in drug side effects. Glucuronidation, which participates in the catabolic process of SN-38 is thus proposed to participate to this interindividual variation and the UGT1A9 enzyme would be responsible, at least in part, for these glucuronidation variations. As example, the UGT1A9(C³Y) and UGT1A9(M³³T) Isoforms, trivially named UGT1A9*2 and UGT1A9*3, respectively, were shown to have a significantly -reduced glucuronidation efficiency toward SN-38 (see Table 1). Therefore, individuals that hold one of these polymorphic variations would be more susceptible to present such adverse physiological reactions.

A person skilled in the art will understand that the invention is not limited to adverse physiological reactions to camptothecin analogs but rather finds uses in the determination of predisposition to physiological reactions to any other glucuronidated compound. Clinically and toxicologically important compounds include mycophenolic acid (MPA), flavopiridol, an anticancer agent under development and a number of xenobiotics, particularly a variety of pre-carcinogens such as the benzo(a)pyrene-trans-7,8-dihydrodiol (BPD), precursor to the potent mutagen benzo(a)pyrene-7,8-dihydrodiol-9,10-epoxide. Glucuronidation is an effective transforming pathway of pyrene to the 1-pyrenylglucuronide, a well-known urinary biomarker for the assessment of human exposure to polycyclic aromatic hydrocarbons. In addition, some UGT isoforms, such as UGT1A9, play a critical role in the detoxification of food-borne carcinogenic heterocyclic amines. Among those, 2-amino-1-methyl-6-phenylimidazo[4,5-b]pyridine (PhlP), the most abundant carcinogenic heterocyclic amine found in well-cooked meats, has been shown to be extensively glucuronidated by UGT1A9 in humans. Genetic polymorphisms is a possible determinant factor of detoxifying UGT1A9 activity and the large interindividual variability in the metabolism of these carcinogens and therapeutics drugs. Finally, a skilled artisan will understand that the present invention also concerns endogenously produced compounds that include, but are not limited to steroids, hormones, fatty acids or bilirubin.

The method of the present invention may further comprise a step of obtaining a nucleic acid sample from the individual and/or extracting nucleic acid material from the biological sample. In such cases, the nature of the biological sample may be adapted for the purpose of the determination and may include saliva, semen, blood, hairs or any specimen comprising at least one cell from a human origin. This specimen can be collected directly on a human body or, alternatively, on any object on which nucleic acid molecules from a human origin could be found. The latter option is of particular interest in cases where inter-generation transmission of a gene (pedigree) is investigated, some members of the cohorts having disappeared. Nucleic acid extraction may include a further step of amplification to ensure an appropriate availability of material, wherein said amplification is preferably performed by polymerase chain reaction (PCR) amplification, wherein PCR amplification is performed using primers that specifically hybridize to a UGT1A9-encoding nucleic acid sequence. Nucleic acid molecules can be either single strand (ss) or double strand (ds) RNA or DNA, as well as DNA/RNA hybrid molecules. In the presence of ssRNA, a step of reverse transcription of the RNA molecule can be performed prior to PCR amplification.

One embodiment of the present invention is to determine the genetic profile of an individual or a patient comprising determining the presence of at least one polymorphic or haplotypic variation in UGT genes. The UGT1A1, UGT1A7 and UGT1A9 genes are the preferred candidate genes according to the present invention, where haplotypic variations can be found in a specific gene or considered simultaneously on multiple genes.

The putative UGT1A9 variations which can be investigated to determine a predisposition to a physiological reaction are C⁻²²⁰⁸T substitution, C⁻²¹⁵²T substitution, C⁻²¹⁴¹T substitution, T⁻¹⁸⁸⁷G substitution, T⁻¹⁸¹⁸C substitution, C⁻⁶⁶⁵T substitution, T⁴⁴⁰C substitution, C⁻³³¹T substitution, T²⁷⁵A substitution, G⁻⁸⁷A substitution, G⁸A missence mutation (C³Y), a T⁹⁸C missence mutation (M³³T), or a combination of these variations. The G⁸A missence mutation is generally associated with a decreased predisposition or susceptibility to an anti-cancer agent whereas the T⁹⁸C missence mutation is associated with an increased predisposition or susceptibility to the same anti-cancer agent. Mutations that can be determined in the UGT1A7 gene are G³⁵³T missense mutation (G¹¹⁵S), T³⁹⁷G missense mutation (N¹²⁹K), C⁴⁰¹A and G⁴⁰²A missense mutations (R¹³K), G⁴²⁷C missense mutation (E¹³⁹D) or T⁶³²C missense mutation (W²⁰⁸R), while the UGT1A1 variation is a TA₇ mutation in the TATA box. A person skilled in the art will recognize that any polymorphic or haplotypic variation found in a UGT gene that modify the expression of the UGT protein, its stability, its substrate specificity, its glucuronidation kinetic parameters or its primary, secondary, tertiary or quaternary structures also represents an aspect of the present invention.

The analysis of a nucleic acid molecule to identify a polymorphic or haplotypic variation can be performed by Restriction Fragment Length Polymorphism (RFLP) analysis, Allele Specific Oligonucleotide (ASO) analysis, Allele Specific PCR (ASP) analysis, Single Strand Conformation Polymorphism (SSCP) analysis, electronic microchip assay, denaturing high-performance liquid chromatography (DHPLC), allelic discrimination assays (Taqman), sequencing or using a DNA chip-based genotyping method, among others.

In one embodiment of the present Invention, the analysis for determining a predisposition or a susceptibility to a drug, as for example but not limited to, an anti-cancer agent in a patient may be restrained to the analysis of UGT1A9 polymorphisms or combined with the analysis of other genes susceptible to lead to a predisposition or susceptibility to the anti-cancer agent (haplotype analysis). The latter genes may encode other drug-conjugating enzymes, such as UGT enzymes as described hereinabove, enzymes that mediate the bioconversion of the CPT-11 molecule into SN-38 (carboxyesterase) or transport enzyme.

Since UGT1A1, UGT1A6, UGT1A7, UGT1A8 and UGT1A10 are the other UGT enzymes that conjugate CPT-11 and SN-38 molecules, the genes that encode these enzymes are targets used to investigate the glucuronidation haplotype of an individual, where at least one of these genes is analyzed concomitantly to UGT1A9. Polymorphic variations in other conjugating enzymes, belonging to the class of carboxyltransferases, sulfotransferases, glutathione S-transferase, methyltransferases or arylamine N-acetyltransferases, β-glucuronidases could also be investigated in concomitance to the UGT1A9 gene.

The transport enzymes described herein include, but are not limited to, ATP-binding cassette (ABC) proteins ABCA1, ABCA2, ABCA3, ABCA4, ABCA5, ABCA6, ABCA7, ABCA8, ABCA9, ABCA10, ABCA11, ABCA12, ABCA13, ABCA14, ABCB1, ABCB2, ABCB3, ABCB4, ABCB5, ABCB6, ABCB7, ABCB8, ABCB9, ABCB_{10,} ABCB1, ABCC1, ABCC2, ABCC3, ABCC4, ABCC5, ABCC6, ABCC7, ABCC8, ABCC9, ABCC10, ABCC11, ABCC12, ABCC13, ABCD1, ABCD2, ABCD3, ABCD4, ABCE1, ABCF1, ABCF2, ABCF3, ABCG1, ABCG2, ABCG4, ABCG5, ABCG8, Breast cancer resistance protein (BCRP), multi-drug resistance protein (MRP) and PGY proteins.

As DNA repair mechanisms could be implicated in the hypersensitivity to camptothecin analogs, haplotype analysis that investigate these mechanism concomitantly to UGT haplotyping analysis is also one embodiment of the present invention. Genes that encode for DNA mismatch repair (MMR), homologous recombination (HR), non-homologous end joining (NHEJ) and single-strand annealing (SSA) systems, as well as Rad and ATPase proteins could therefore be analyzed by a skilled artisan simultaneously to UGT sequences.

In a further embodiment of the present invention, there is provided an isolated nucleotide molecule comprising an allelic variant of a polymorphic region of a UGT1A1 gene, wherein -the allelic variant comprises at least one TATA box polymorphic variation within the UGT1A1 promoter region.

According to another embodiment of the present invention, there is provided an isolated nucleotide molecule comprising an allelic variant of a polymorphic region of a UGT1A7 gene, wherein the allelic variant comprises at least one nucleotide sequence selected from the group consisting of those set forth in SEQ ID No: 60 to SEQ ID NO: 68, or the complement thereof.

Also, there is provided an isolated nucleotide molecule comprising an allelic variant of a polymorphic region of a UGT1A9 gene, wherein the allelic variant comprises at least one nucleotide sequence selected from the group consisting of those set forth in SEQ ID NO: 36 to SEQ ID NO: 59, or the complement thereof.

In a further embodiment, there is provided an isolated amino acid sequence comprising at least one amino acid sequence selected from the group consisting of SEQ ID NO: 69, SEQ ID NO: 70, SEQ ID NO: 71 or a fragment thereof. These amino acid sequences may be encoded by a nucleotide sequence comprising at least one sequence selected from the group consisting of SEQ ID NO: 36, SEQ ID NO: 37, SEQ ID NO: 38, a fragment or the complementary sequences thereof. Alternatively, the expression of the amino acid sequence may be regulated by a nucleotide sequence comprising at least one sequence selected from the group consisting of SEQ ID NO: 39, SEQ ID NO: 40, SEQ ID NO: 41, SEQ ID NO: 42, SEQ ID NO: 43, SEQ ID NO: 44, SEQ ID NO: 45 SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, a fragment or the complementary sequences thereof.

The present invention will be more readily understood by referring to the following examples which are given to Illustrate the invention rather than to limit its scope.

### EXAMPLE I

### Distribution of SN-38-Glucuronide Formation In Human Liver Microsome Samples

To obtain statistical data on interindividual variation of SN-38 glucuronidation, we measured the SN-38-G formation, as currently known in the background art, with microsomes preparations from each patient liver sample. The glucuronide formation rates were regrouped into ranges and every sample was categorized within these ranges.

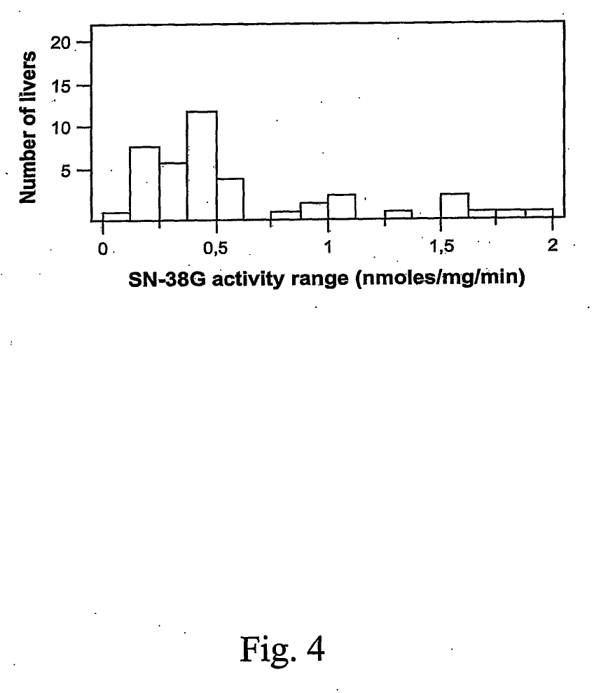

The following results show a mean for glucuronidation rate of 0.61 pmol/mg of protein/minute (Table 1). Data also indicate a substantive distribution of the glucuronidation rates. FIG. 4 illustrates the distribution of the glucuronidation rates obtained with liver samples.

TABLE 1
Statistical data of SN-38 glucuronide formation by human liver samples

| Quantiles | | | Moments | |
|---|---|---|---|---|
| | | | Mean | 0.6117859 |
| 100.0% | maximum | 1.9735 | Std Dev | 0.5014612 |
| 99.5% | | 1.9735 | Std Err Mean | 0.0723797 |
| 97.5% | | 1.9309 | upper 95% Mean | 0.7573947 |
| 90.0% | | 1.5699 | lower 95% Mean | 0.4661772 |
| 75.0% | quartile | 0.9279 | N | 48 |
| 50.0% | median | 0.4204 | Sum Wgts | 48 |
| 25.0% | quartile | 0.2763 | Sum | 29.365725 |
| 10.0% | | 0.1641 | Variance | 0.2514633 |
| 2.5% | | 0.1113 | Skewness | 1.3394982 |
| 0.5% | | 0.1063 | Kurtosis | 0.6950696 |
| 0.0% | minimum | 0.1063 | CV | 81.966773 |

### EXAMPLE II

### Identification of UGT1A9 Variants

Material and Methods

DNA samples

DNA samples of 201 Caucasian subjects were obtained from the Quebec Family Study (QFS) (Simonen et al., 2002, Med. Sci. Sports Exerc. 34: 1137-1142). Unrelated Caucasian subjects were recruited at the Massachusetts General Hospital (n=100) and genomic DNA from African-American subjects were kindly provided by Robert Millikan (Lineberger Comprehensive Cancer Center, School of Medicine, University of North Carolina, Chapel Hill, N.C. 27599-7435, USA) (n=20). These samples had been anonymized prior to their reception in our laboratory. All subjects have provided written consent for the use of their DNA for experimental purposes, and the present study was reviewed and approved by Institutional Review Boards (CHUL Research Center is and Laval University).

Resequencing of the UGT1A9 Gene and Genotyping

Polymerase chain reaction (PCR) was used to amplify the first exon of the UGT1A9 gene. Three pairs of primers were designed to amplify overlapping fragments covering the first exons, a small portion of the 5′-flanking region and the intron-exon boundary (listed in Table 1). PCR amplification and DNA sequencing were performed according to protocols of Faucher et al (Faucher et al., 2002, Hum. Mol. Genet. 11: 2077-2090). Amplicons were sequenced with an ABI 3700™ automated sequencer using Big Dye™ (Perkin Elmer™) dye primer chemistry. Samples were sequenced on both strands with nested primers listed in Table 2. Samples with ambiguous sequencing chromatograms and samples with single nucleotide polymorphisms (SNPs) were subjected to a second, independent amplification, followed by DNA sequencing. Sequences were analyzed with Staden preGap4 and Gap4 programs. These programs align sequence chromatograms and Identify areas in which polymorphisms might be present. Each chromatogram was then evaluated individually to confirm variation in the sequences.

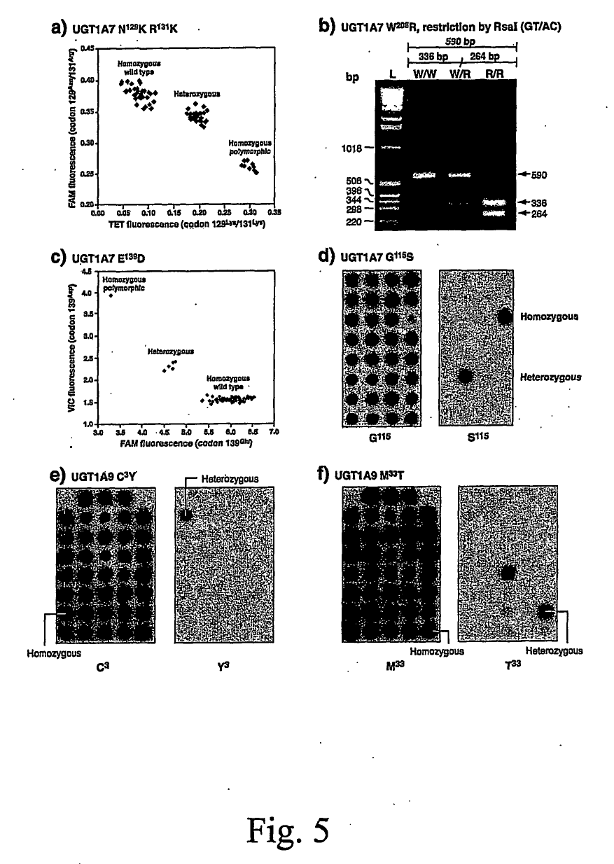

To determine the prevalence of UGT1A9 alleles in the population, a portion of the first exon, which includes the newly discovered polymorphisms, was amplified by PCR using specific oligonucleotides #37 and #38 (SEQ ID NO:. 1 and 2). PCR amplifications were performed in a final reaction volume of 50 μL containing 25 ng of genomic DNA, 20 pmol of each primer, 1× reaction buffer, 100 μM dNTPs, 4% DMSO and 2 U of the Taq DNA polymerase. The amplification conditions were: denaturation at 96° C. for 5 min, 35 cycles of 30 sec at 94° C., 40 sec at 58° C. and 1 min at 72° C., with a final extension step of 7 min at 72° C. Reactions were performed in a Perkin Elmer™ model 9700 thermal cycle. ASOs were designed to detect by hybridization the missense mutations in the UGT1A9 amplification products. Four ASOs were designed to specifically hybridize to the sequence corresponding to a G or an A at codon 3 (FIG. 5e) and a T or a C at codon 33 (FIG. 5f) and hybridization performed as previously described (Guillemette et al., 2000, Pharmacogenetics 10: 629-644).

TABLE 2
Primer sequences for UGT1A9

| | | |
|---|---|---|
| | | SEQ ID |
| Primers | Sequences | NO: |

| | | |
|---|---|---|
| PCR amplification UGT1A9 | | |
| #37 | 5′-gtgctggtatttctccc | 1 |
| #38 | 5′-gtcaaaaatgtcattgtatgaacc | 2 |
| #39 | 5′-gatctggaccgggagttcaa | 3 |
| #40 | 5′-gtgtggctgtagagatcatact | 4 |
| #41 | 5′-catgcacttggaggaacatttatta | 5 |
| #42 | 5′-gagtacacgcattggcac | 6 |
| Direct sequencing UGT1A9 | | |
| #7 | 5′-ctcccacctactgtatc | 11 |
| #8 | 5′-gttcaaggcttttgccc | 12 |
| #9 | 5′-catttattatgccaccg | 13 |
| Allelic specific oligos UGT1A9 | | |
| C³ | 5′-atggcttgcacagggt | 14 |
| Y³ | 5′-atggcttacacagggt | 15 |
| M³³ | 5′-agtgcccatggatggga | 16 |
| T³³ | 5′-agtgcccacggatggga | 17 |
| Site-directed mutagenesis UGT1A9 | | |
| C⁸ to Y³ (Forward) | 5′-gttctctgatggcttacacagggtggaccag | 28 |
| C³ to Y³ (Reverse) | 5′-ctggtccaccctgtgtaagccatcagagaac | 29 |
| M³³ to T³³ (Forward) | 5′-gctactggtagtgcccacggatgggagccactgg | 30 |
| M³³ to T³³ (Reverse) | 5′-ccagtggctcccatccgtgggcactaccagtagc | 31 |

Bold: nucleic acid polymorphism

Methods for UGT1A9 SNPs Detection

UGT1A9 first exon was amplified in unrelated subjects. Allelic discrimination PCR was used to genotype UGT1A9 codons 3 and 33. The probe marked with FAM fluorochrome was designed to detect the wild type allele. The other probe used to detect the polymorphic alleles were marked with TET fluorochrome.

Duplicate filters were hybridized separately with the corresponding γ-³²P labeled oligonucleotides. The positive signals detected with both ASOs indicated heterozygous individuals for the polymorphism in contrast with a positive signal with one probe only, which indicated that the subject was homozygous.

Identification of Missense Mutations in the Human UGT1A9 First Exon by Direct Sequencing of PCR Products.

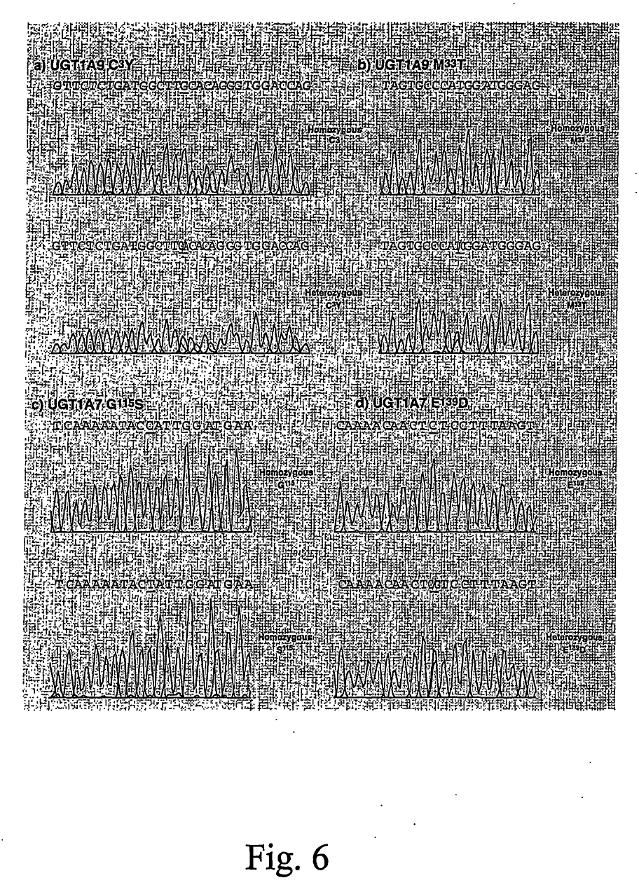

Specific primers were used to amplify the exon 1 UGT1A9 (SEQ ID NO: 1 to SEQ ID NO: 6). The nonsynonymous polymorphisms in the third codon (C³Y) (a) and in codon 33 (M³³T) of UGT1A9 (b) are illustrated In FIG. 6.

Functional Analysis of the Conjugating Activity of UGTA9 Variants

Microsomal fractions from HEK-293 cells stably expressing human UGT1A9*1, UGT1A9*2 and UGT1A9*3 were used in enzymatic assays. Reactions (100 μl volume) contained 50 mM Tris-HCl, pH 7.3, 10 mM MgCl₂, 100 μg/mL phosphatidylcholine, 1 mM UDP-glucuronic acid, 40 to 60 μg of membrane protein. SN-38, MPA or other substrates were added in concentrations ranging from 1 to 200 μM and the reaction was incubated 30 min. at 37° C. with agitation. Human liver microsome were incubated in the same condition for control. Reaction was stopped by the addition of 200 μL MeOH+1% HCl 2N, followed by centrifugation at 14000 rpm for 10 minutes. Supernatant was filtered through a 0.22 μm filter and 100 μL of water was added. For SN-38 and SN-38-glucuronide detection, 10 μL samples were injected on a liquid chromatographic system coupled to a fluorescence detector. Time-course experiments were realized to determine the linearity of the glucuronidation reaction. For determination of Vₘₐₓ and Kₘ, HEK-293 cells stably expressing UGT enzymes were incubated in the presence of varying SN-38 concentrations from 1 to 200 μM for the corresponding period of 30 min. All reactions rates were shown to be linear for these times.

A liquid chromatographic method was developed to quantify SN-38 glucuronidation of UGT cell line-derived microsomes and human liver microsomes. Samples were analyzed using high performance liquid chromatography (Alliance 2695, Waters, Milford, Mass.). Chromatographic separation was achieved with a Colombus C18 column 5-μm packing material, 50×3.2 mm (Phenomenex, Torrance, Calif.) using a two-solvent gradient system: A (water+1 mM ammonium formate); B (MeOH+1 mM ammonium formate). At a constant flow rate (0.7 ml/min), a linear gradient from 20 to 65% B was run over 3 min, held 0.8 min and a second gradient until 95% of B was run over 2 min and then re-equilibrated to 20% B over 2 min. The effluent from the HPLC system (Alliance 2695) was connected directly to a fluorescence detector (Waters, Milford, Mass.) using an excitation wavelength of 460 nm and an emission of 460 nm. Retention time for SN-38 and SN-38-glucuronide were respectively 3.5 and 4.6 min. Determination of the glucuronidation rates obtained with other substrates was performed as currently known in the art.

Results

Identification of Two Novel Missense Mutations in the Human UGT1A9 Gene and Their Distribution in Healthy Individuals.

The strategy used to identify polymorphisms in the UGT1A9 gene was a PCR amplification of the exon 1, followed by direct DNA sequencing. Inclusion of a portion of the adjacent intron and 5′-flanking region in the PCR fragment was performed in order to assure the specific amplification of the UGT1A9 gene. The UGT1A9 was resequenced on both strands for 35 subjects. DNA samples from Caucasian-American subjects was shown to contain one SNP, whereas an additional SNP was observed in an African-American subject. No insertion-deletion events were observed within the area sequenced.

The nucleotide change producing the first cSNP (SNPs in the coding region) was a change of a G to an A at nucleotide 8. The polymorphic change results in the substitution of Cysteine by a Tyrosine (C³Y) in the signal peptide of the UGT1A9 protein corresponding to the UGT1A9*2 allele (SEQ ID NO: 37). The second nucleotide change, T⁹⁸C, leads to a Methionine to a Threonine at codon 33 (M³³T) corresponding to the UGT1A9*3 allele (SEQ ID NO: 38). FIGS. 6a and 6b illustrate the sequence analysis of three genotypes: homozygous wild type *1/*1 and heterozygous *1/*2 or *1/*3.

To determine the allelic frequency of UGT1A9 allozymes in the population, we genotyped unrelated subjects including 301 Caucasians of whom 201 were French-Canadians, and 20 African-American subjects. Only one African-American individual had the C³Y mutation whereas 12 individuals, all Caucasian subjects, were shown to have the M³³T mutation (illustrated in FIG. 5f). A total of 5% of individuals were found heterozygous for the UGT1A9*3 allele in the French-Canadian population and 3% of the remaining Caucasian-American subjects. None of the 20 African-American subjects were found to have the UGT1A9*3 allele (Table 3).

TABLE 3
Allelic frequency and prevalence of UGT1A9 alleles

| | | | | | Genotype | | |
|---|---|---|---|---|---|---|---|
| | Allele frequency | | | | frequency (%)^{a} | | |
| | n | *1 | *2 | *3 | *1/*1 | *1/*2 | *1/*3 |
| Amino acid change | | Cys³Met³³ | Tyr³Met³³ | Cys³Thr³³ | | | |
| Functional change | | “Wild type” | Similar | Decreased | | | |
| | | | activity | activity | | | |
| Population characteristics | | | | | | | |
| Caucasian (French Canadian) | 201 | 0.978 | 0.000 | 0.022 | 95 | 0 | 4.4 |
| Caucasian (American) | 100 | 0.964 | 0.000 | 0.036 | 97 | 0 | 3 |
| African (American) | 20 | 0.975 | 0.025 | 0.000 | 95 | 5 | 0 |

^{a}Subjects homozygous for variant UGT1A9 alleles were not observed in the population tested.

Functional Analysis of the Conjugating Activity of UGTA9 Variants

Table 4 shows that the presence of a threonine at position 33 (UGT1A9*3) is correlated to 96.3% decreased conjugation rate for SN-38 while the presence of a tyrosine at codon 3 is associated to a 16.7% increased activity. Moreover, modulation of the UGT1A9 glucuronidation activity is substrate specific since conjugation of eugenol, 2-hydroxyestradiol, 4-hydroxyestrone and 4 methylumbelliferone is increased or decreased in a proper way for each substrate. The presence of a threonine at position 33 does not affect significantly the affinity of the protein for SN-38 but decreases by approximately 20-folds its glucuronidation rate (Table 5) while the affinity of UGT1A9 for MPA is dramatically reduced by the presence of codon 33 variation (Table 6).

TABLE 4
Substrate-dependent modulation of the UGT1A9
activity by codons 3 and 33.

| | % glucuronide formation relative to | |
|---|---|---|
| | UGT1A9*1 | |
| Substrates | UGT1A9*2 | UGT1A9*3 |
| Eugenol | ↓28.4% | ↑716% |
| 2-OH-E2 | ↓26.6% | ↑1727% |
| 4-OH-E1 | ↓19.0% | ↓90.3% |
| 4-MU | ↓19.2% | ↓66.2% |
| SN-38 | ↑16.7% | ↓96.3% |
| Flavopiridol | ns | ns |

TABLE 5
Kinetic analysis of SN-38 glucuronidation by UGT1A9*1, *2 and *3

| | 1A9*1 | 1A9*2 | 1A9*3 |
|---|---|---|---|
| Km | 3.03 ± 0.72 | 5.15 ± 1.81 | 3.21 ± 0.95 |
| Vmax | 316.34 ± 52.03 | 324.68 ± 95.09 | 15.50 ± 8.40 |
| | | | p < 0.001 |
| Vmax/Km | 104 | 63 | 5 |
| (CIᵢₙₜ) | | | |

TABLE 6
Kinetic analysis of MPA glucuronidation by UGT1A9*1, *2 and *3

| UGT1A9 | Km | Vmax | |
|---|---|---|---|
| alleles | μM | pmol/min/mg | Vmax/Km |
| 1A9*1 | 495 | 9406 | 19 |
| 1A9*2 | 303 | 8401 | 28 |
| 1A9*3 | 3225 | 14074 | 4 |

### EXAMPLE III

### Identification of Novel UGT1A9 Promoter Variants

The primary objective of this study was to examine the genomic sequences of the UGT1A9 gene promoter sequence to Identify novel expression polymorphisms and to determine whether or not these polymorphic variations would affect the expression of the UGT1A9 protein. To determine the effect of the polymorphic variations on the UGT1A9 protein expression, semi-quantitative immunoblot analyses were- performed on liver microsomes from patients and correlated with their genotypes. Identification of novel polymorphisms has been performed by direct sequencing of a pool of DNA samples from patients. Determination of genotypes of each patient monitored was also performed by direct sequencing.

Liver microsomes from patients were prepared by differential centrifugation. The crude cell extracts were centrifuged at 12000×g at 4+ C. for 22 min to remove nuclei and other cellular debris. Supernatants were centrifuged at 105000×g for 60 min at 4° C. to obtain the membrane fraction, which was homogenized in the buffer described above. Protein concentrations were determined using the Bradford method according to the manufacturers recommendations.

To determine the level of UGT1A9 proteins expressed in the microsomal fractions obtained from liver microsomes, Western blot analyses were conducted as follows: Microsomal proteins (10 μg) from liver microsomes were separated by 10% SDS-polyacrylamide gel electrophoresis. The separated proteins were transferred onto nitrocellulose membranes and probed with the antihuman UGT1A antiserum (1:1000 dilution) specific for the amino-terminal region of the UGT1A7, UGT1A8, UGT1A9 and UGT1A10 proteins. Given that UGT1A7, UGT1A8 and UGT1A10 are not expressed in liver tissue, immunodetection with this antiserum in human liver microsomes is specific to UGT1A9. In order to normalize sample loading, blots were re-probed with anti-calnexin antibody (1:2000 dilution; StressGen Biotechnologies Corp., Victoria, Canada), to detect a second ER-resident protein. A donkey antirabbit IgG antibody conjugated with the horseradish peroxidase (Amersham Corp., Oakville, Canada) was used as the secondary antibody (1:10000 dilution). The resulting immunocomplexes were visualized using an enhanced chemiluminescence kit (ECL) (Renaissance, Quebec, Canada) and exposed on Kodak XB-1 film. The lowest signal has been used as standard to determine the relative expression of UGT1A9 in each sample and results were monitored by Oneway analyses.

Results

Ten novel polymorphic variations were Identified within the UGT1A9 promoter region, namely a C(−2208)T substitution, a C(−2152)T substitution, a C(−2141)T substitution, a T(−1887)G substitution, a T(−1818)C substitution, a C(−665)T substitution, a T(−440)C substitution, a C(−331)T substitution, a T(−275)A substitution and a, G(−87)A substitution.

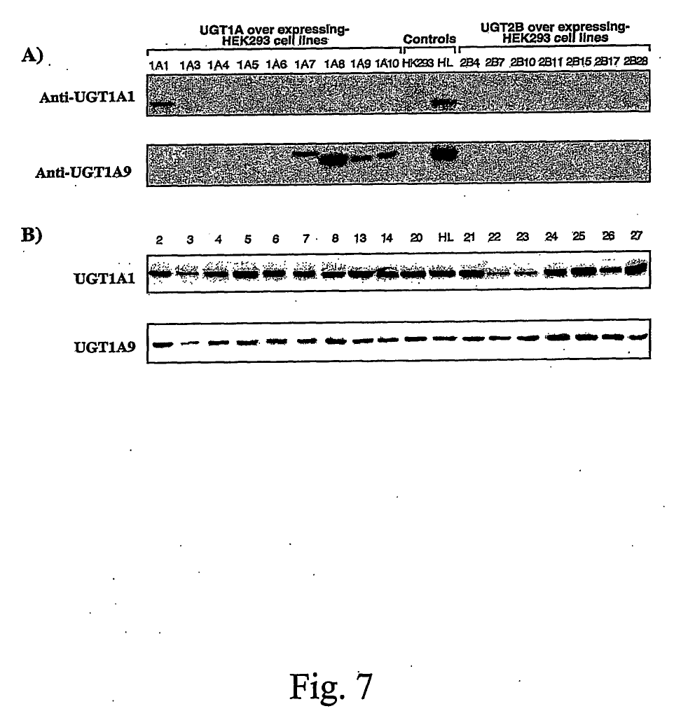

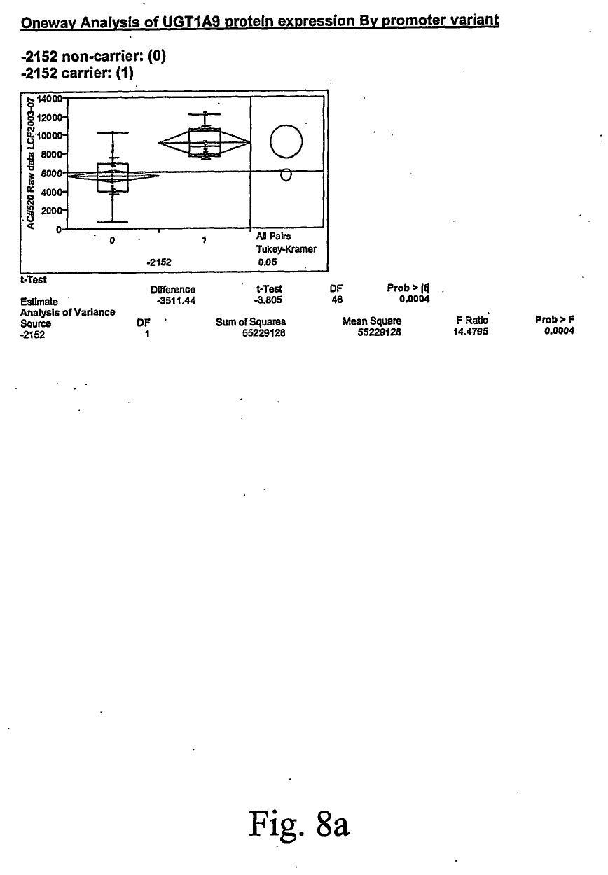

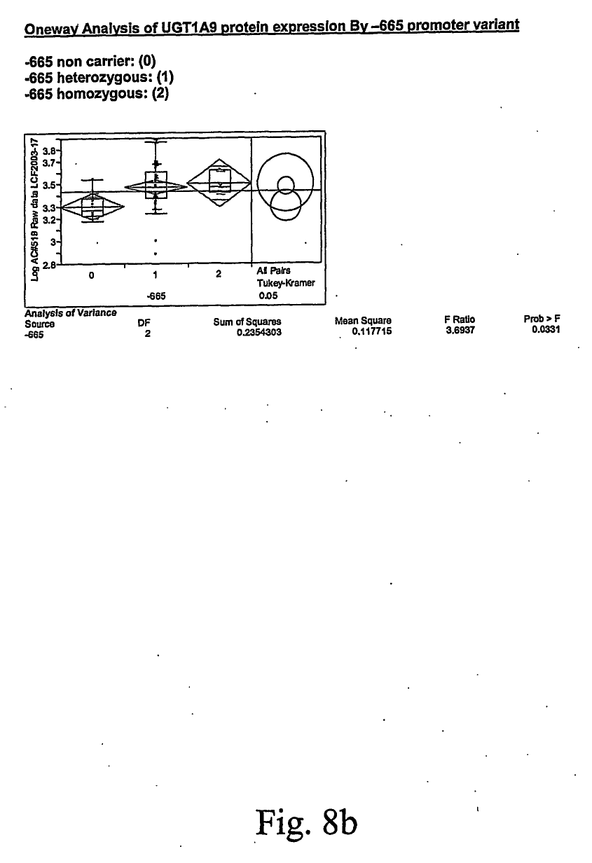

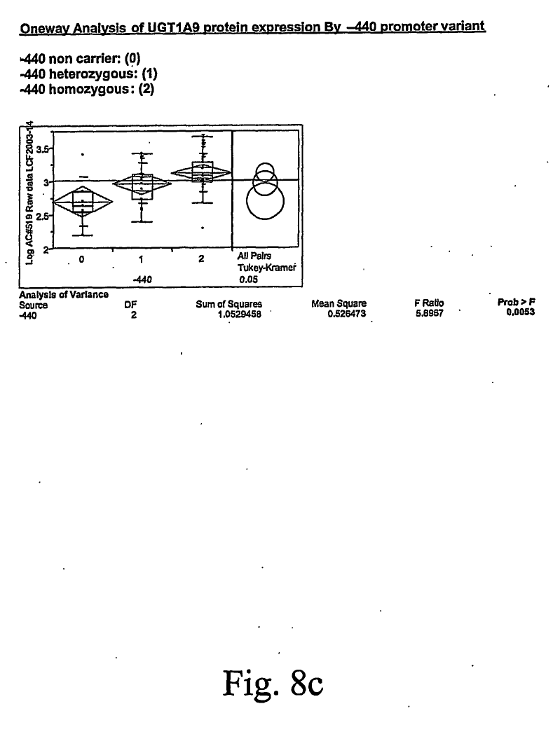

UGT1A9 protein expression is highly variable among tested samples, as shown on FIG. 7. FIGS. 8a to 8e demonstrate a positive correlations between the presence of mutated nucleic acids in positions −2152 (FIG. 8a), −665 (FIG. 8b), and −440 (FIG. 8c) in the promoter region of the UGT1A9 gene and the expression of higher level of UGT1A9 proteins.

### EXAMPLE IV

Effect of UGT1A9 Polymorphic Variations on Liver Microsomes Glucuronidation

One it has been established that polymorphic variations in the promoter region of the UGT1A9 gene can modulated the expression of the UGT1A9, it was interesting to study the impact of these mutation on global glucuronidation by human liver microsomes. Therefore, a correlation study was undergone to determine if correlations could exist between C(−2152)T. T(−1818)C, C(−665)T and T(−275)A variations and SN-38, mycophenolic acid and 4hydroxyestrone glucuronide formation. Glucuronidation activity was determined for each liver sample in nmoles/mg of proteins/min and further regrouped respective to the genotype of the patient, namely patient carrying a mutation or non carrying (wild type) patients.

Results

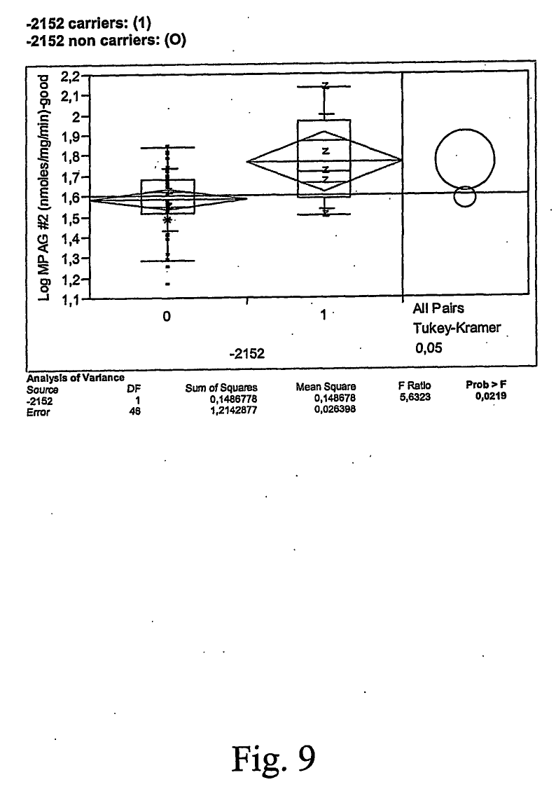

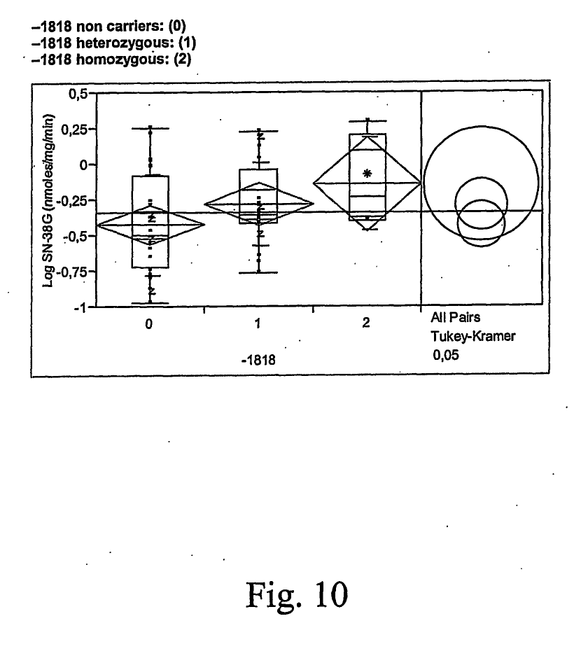

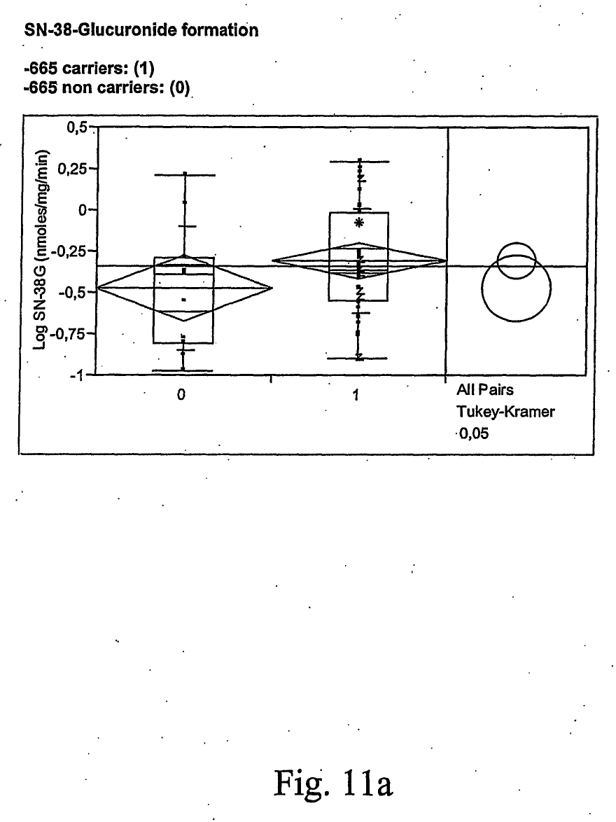

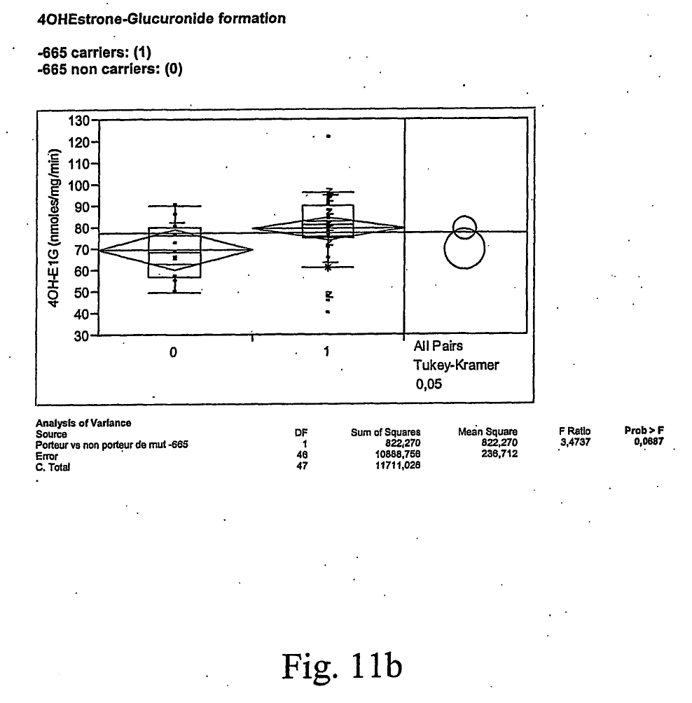

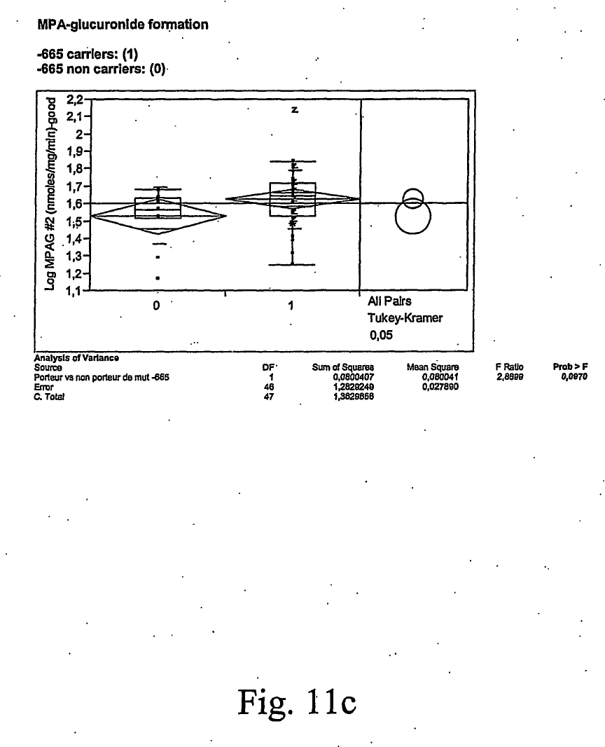

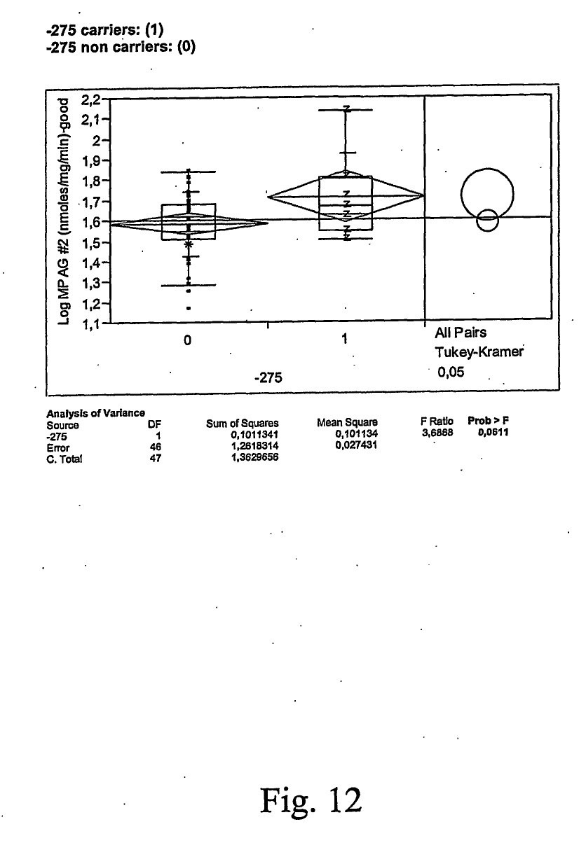

One way analyses demonstrate a correlative association between the presence of a mutated nucleic acid at position −2152 and glucuronidation of MPA (FIG. 9). FIG. 10 also shows a positive correlation between the formation of SN-38-glucuronide and the presence of one or both mutated alleles at position −1818 in the UGT1A9 promoter region. Nucleic acid change at position −665 correlates with higher glucuronidation rates with SN-38, (FIG. 11a), 4-hydroxyestrone (FIG. 11b) and mycophenolic acid (FIG. 11c). Finally, FIG. 12 shows a positive correlation between the presence of the −275 mutated alleles and higher glucuronidation rate with SN-38.

### EXAMPLE V

### Effect of the Expression of UGT1A Proteins on Glucuronidation by Liver Microsomes

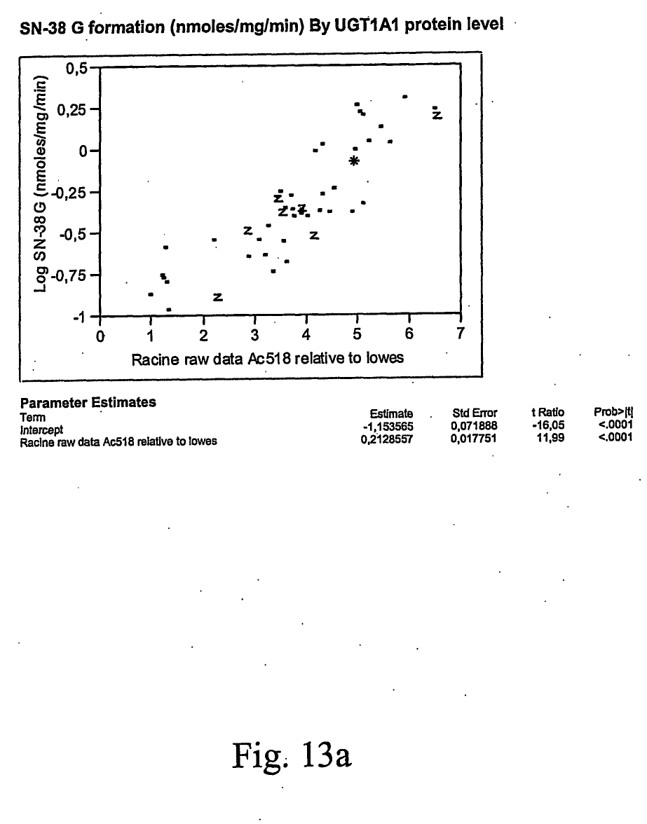

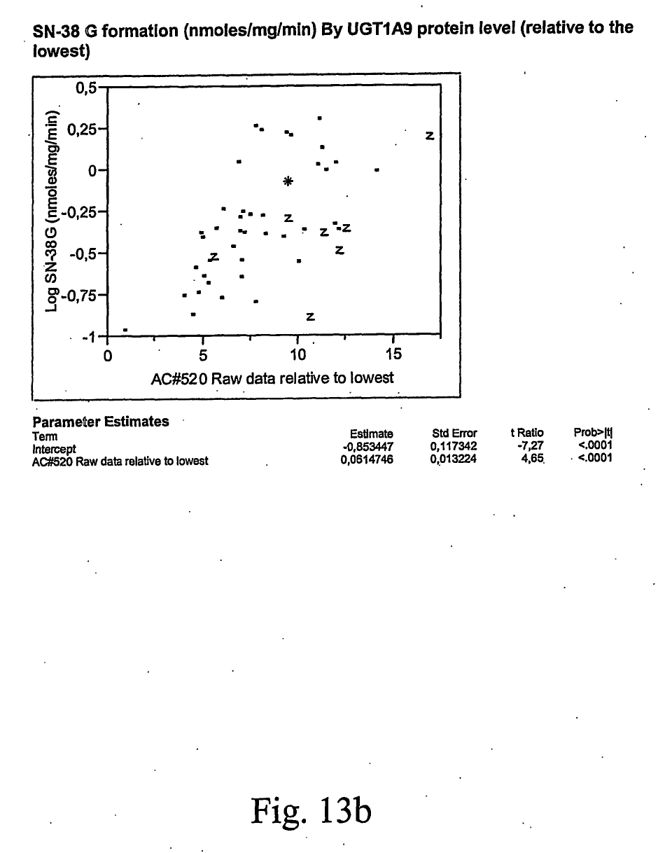

As UGTA9 is considered as a major SN-38 glucuronidation enzyme, we attempted to determine if an association between the expression of this proteins and glucuronide formation could exist. As shown in FIG. 13a, there is a positive correlation between glucuronidation of SN-38 and protein level of UGT1A9. To ascertain that the enhancement of glucuronidation observed with this substrate is not attributable to a residual activity of other UGT isoforms, these experiments were reconducted using a probe substrate for UGT1A9, namely mycophenolic acid. FIG. 13b illustrates the positive correlation between UGT1A9 protein expression level and MPA glucuronidation.

### EXAMPLE VI

### Identification of Novel UGT1A7 Variants

The primary objective of the study was to examine the genomic sequences of the UGT1A7 gene, for which functional polymorphisms have been described yet to identify novel polymorphic variations. The aim was to look for missense polymorphisms in a Caucasian population, to develop methods for SNPs detection and to evaluate their functional properties after In vitro expression of enzyme variants. In turn, UGT1A7 is a polymorphic gene for which there are at present four known allelic variants (Guillemette et al., 2000, Pharmacogenetics, 10: 629-640). Based on In vitro metabolic studies, the UGT1A7*3 and *4 variants may potentially lead to a poor SN-38 glucuronidator phenotype.

Material and Methods

UGT1A7 Haplotype Determination

DNA samples were obtained according to Example 2. To discriminate the polymorphisms at codons 129/131 and 139, a PCR technique using the Taqman® technology was used (Applied Biosystems, Branchburg, N.J., USA). To discriminate the two alleles at codons 129/131, the exon 1 containing the codon 129/131 was amplified using primers 387 and 388 (SEQ ID NO: 20 and 21, respectively) shown in Table 4. Two probes were designed to identify the two different alleles, probe for N¹²⁹/R¹³¹ allele was marked with FAM fluorochrome and probe for K¹²⁹/K¹³¹ allele was marked with TET fluorochrome. Also, specific primers were designed to amplify the region of exon 1 containing codon 139. Specific 21-mer probes were designed to identify the two different alleles. One of the probes, E³⁹-FAM, was homologous to the wild type allele. The other probe, D¹³⁹-VIC, contained the polymorphic nucleotide at codon 139 in order to be homologous to the D¹³⁹ mutant allele. Each PCR reaction was performed with 25 ng of genomic DNA in a volume of 10 μL and containing 5 pmole of each primer and probe and 1×Taqman® universal PCR master mix. PCR conditions were 50° C. for 2 minutes, 95° C. for 10 minutes followed by 40 cycles at 95° C. for 16 seconds and 60° C. for 1 minute. The ABI prism 7000™ system detected the different genotypes (FIGS. 5a; 5c).

The polymorphism at codon 208 of UGT1A7 was genotyped by PCR-RFLP. The polymorphism at codon 208 creates a restriction site for Rsa I enzyme. Digestion was performed with 5 μL of PCR product, 10 U of Rsa I and 1×reaction buffer L (10 mM Tris-HCl, 10 mM MgCl₂, 1 mM DTE, PH 7.5) in a total volume of 10 μL. Reactions were incubated for 2 hours at 37° C. and separated on a 2% agarose gel to observe the different migration pattems. Homozygous wild type genotype at codon 208 generates a single fragment migrating at 590 bp. The heterozygous genotype generates a fragment of 590 pb representing the wild type allele and two bands of 236 and 264 bp representing the polymorphic allele cut by Rsa I. Homozygous mutants at position 208 have a pattern of migration showing only two bands of 236 and 264 bp (FIG. 5b).

Allelic specific oligonucleotides (ASOs) were designed to detect UGT1A7 polymorphism at codon 115. PCR amplification using primers 292 and 293 was used to generate the target fragment containing the polymorphic site. Each ASO is composed of a 17-mer centered over the polymorphic nucleotide of each variant. The denatured PCR products were spotted onto filters, each one being subsequently hybridized with a single ASO using a method that has been described previously (Guillemette et al., 2000, Pharmacogenetics, 10: 629-640). Conditions for ASO hybridization analysis have been described above and a typical result is illustrated in FIG. 5d.

Methods for UGT1A7 SNPs Detection.

UGT1A7 first exon was amplified in unrelated subjects. (a) Allelic discrimination PCR was used to genotype UGT1A7 codons 129/131. The probe marked with FAM fluorochrome was designed to detect the wild type N¹²⁹/R¹³¹ allele. The other probe used to detect the polymorphic allele K¹²⁹/K¹³¹ was marked with TET fluorochrome. (b) PCR products amplified with primers #17 (SEQ ID No: 8) and #18 (SEQ ID No: 7) were digested using Rsa I enzyme to determine whether the patients were homozygous wild type W²⁰⁸, heterozygous W²⁰⁸/R²⁰⁸ or homozygous R²⁰⁸. The 590 bp fragment represents the undigested PCR product whereas the 336 and 264 bp fragments result from the digestion of the 590 bp amplicon. (c) Allelic discrimination PCR was used to genotype the novel polymorphism at codon 139 of the UGT1A7 gene. The FAM fluorochrome was used to mark the wild type probe E¹³⁹ and the VIC fluorochrome was used for the polymorphic probe D¹³⁹. (d) Allelic specific oligonucleotides (ASOs) were designed to genotype the novel polymorphic variation at codon 115 of UGT1A7 gene. (e) (f) A similar strategy was further used to detect variants at codons 3 and 33 of the UGT1A9 gene. Duplicate filters were hybridized separately with the corresponding γ-32P labeled oligonucleotides. The positive signals detected with both ASOs indicated heterozygous individuals for the polymorphism in contrast with a positive signal with one probe only, which indicated that the subject was homozygous.

Identification of Missense Mutations in the Human UGT1A7 First Exon by Direct Sequencing of PCR Products.

Specific primers were used to amplify the exon 1 of UGT1A7 (Table 7). The nonsynonymous polymorphisms illustrated, along with the codon 115 (G¹¹⁵S) (c) and codon 139 (E¹³⁹D) (d) polymorphisms of UGT1A7 (see FIG. 6). The sequence illustrated in (a) and (b) correspond to the “sense” strand whereas (c) and (d) correspond to the “anti-sense” strand.

TABLE 7
Primer sequences for UGT1A7

| | | |
|---|---|---|
| | | SEQ ID. |
| Primers | Sequences | NO: |

| | | |
|---|---|---|
| PCR amplification UGT1A7 | | |
| #18 | 5′-cgctggacggcaccattg | 7 |
| #17 | 5′-gctaaaggggagataacttacc | 8 |
| #122 | 5′-gctggacggcaccattg | 9 |
| #123 | 5′-ccctaagagaagtctgggg | 10 |
| Allelic specific oligos UGT1A7 | | |
| G¹¹⁵ | 5′-catccaatggtattttt | 18 |
| S¹¹⁵ | 5′-catccaatagtattttt | 19 |
| Taqman ® analysis | | |
| (Codon 129/131) UGT1A7 | | |
| #387 | 5′-gcaccattgcgaagtgcat | 20 |
| #388 | 5′-ggatcgagaaacactgcatcaa | 21 |
| N129/R131-FAM | 5′-ttaatgaccgaaaatt | 22 |
| K129/K131-TET | 5′-tttaaggacaaaaaatt | 23 |
| Taqman ® analysis | | |
| (Codon 139) UGT1A7 | | |
| #546 | 5′-gcgaagtgcattttctctattaacaa | 24 |
| #544 | 5′-aagccacagcgatcaaaagg | 25 |
| E139-Fam | 5′-atacttaaaggagagttgttt | 26 |
| D139-Vic | 5′-atacttaaaggacagttgttt | 27 |
| Site-directed | | |
| mutagenesis UGT1A7 | | |
| E¹³⁹ to D¹³⁹ (Forward) | 5′-aattagtagaatacttaaaggacagttgttttgatgcagtgtttc | 32 |
| E¹³⁹ to D¹³⁹ (Reverse) | 5′-gaaacactgcatcaaaacaactgtcctttaagtattctactaatt | 33 |
| G¹¹⁵ to S¹¹⁵ (Forward) | 5′-gttcatccaatagtatttttgac | 34 |
| G¹¹⁵ to S¹¹⁵ (Reverse) | 5′-gtcaaaaatactattggatgaac | 35 |

Bold: nucleic acid polymorphism

Results

Identification of Two Novel Polymorphisms in the Coding Region of the UGT1A7 Gene and Haplotypic Structure Analysis of the UGT1A7 Gene.

The exon 1 of UGT1A7 was amplified by PCR in 117 subjects, 54 Caucasians and 63 African-Americans, and then sequenced. Two novel polymorphisms were found at codon 115 and 139 (FIGS. 6c; 6d). At codon 115, a nucleotide change of a G to an A leads to an amino acid change from Glycine to Serine (G¹¹⁵S). A G to C mutation at codon 139 leads to an amino acid change from Glutamate to Aspartate (E¹³⁹D).

When combined with the previously described variations at codon 129/131 and 208, nine haplotypes were found to exist (UGT1A7*1 to *9, Table 4). Four alleles were previously described, *1 to *4, and novel alleles correspond to UGT1A7*5 S¹¹⁵N¹²⁹R¹³¹E¹³⁹W²⁰⁸ (Genbank AF434903), UGT1A7*6 G¹¹⁵N¹²⁹R¹³¹D¹³⁹W²⁰⁸ (Genbank AF434904), UGT1A7*7 G¹¹⁵K¹²⁹Kg¹³¹D¹³⁹W²⁰⁸ (Genbank AF461758), UGT1A7*8 G¹¹⁵K¹²⁹K¹³¹D¹³⁹R²⁰⁸ (Genbank AF436810) and UGT1A7*9 S¹¹⁵K¹²⁹K¹³¹E¹³⁹W²⁰⁸ (Genbank AF463483).

According to their prevalence in the population tested, the nine variant alleles were separated in two categories: the common and the rare alleles. The common alleles *1, *2 and *3, are present at a allelic frequency of 0.31 to 0.32. The rare alleles are UGT1A7*4 to *9, with frequencies between 0.002 to 0.025. The allelic frequencies for the polymorphisms at codon 115 and 139 were 0.04 and 0.06, respectively and found specifically in African-American individuals.

TABLE 8
Allelic frequency and prevalence of UGT1A7 alleles

| UGT1A7 alleles | | Function^{b} | Frequency^{c} |
|---|---|---|---|
| UGT1A7*1^{a} | | High | 32.18 |
| UGT1A7*2 | K¹²⁹/K¹³¹ | High | 30.60 |
| UGT1A7*3 | K¹²⁹/K¹³¹/R²⁰⁸ | Low | 31.55 |
| UGT1A7*4 | R²⁰⁸ | Low | 2.52 |
| UGT1A7*5 | S¹¹⁵ | Low | 0.47 |
| UGT1A7*6 | D¹³⁹ | High | 0.16 |
| UGT1A7*7 | K¹²⁹/K¹³¹/D¹³⁹ | High | 2.06 |
| UGT1A7*8 | K¹²⁹/K¹³¹/D¹³⁹/R²⁰⁸ | Low | 0.16 |
| UGT1A7*9 | S¹¹⁵/K¹²⁹/K¹³¹ | Low | 0.32 |

^{a}UGT1A7*1: G¹¹⁵/N¹²⁹/R¹³¹/E¹³⁹/W²⁰⁸; only position differing from *1 are indicated
^{b}Based on in vitro experiments: Low: significantly lower SN-38G formation versus *1 allele.
High: no significant difference in activity compared to *1 allele.
^{c}Population of 167 Caucasian and 150 African-American subjects.

TABLE 9
Frequency of the UGT1A7 alleles

| | Population | |
|---|---|---|
| UGT1A7 genotypes^{a} | (n = 317)^{b} | Frequency (%) |
| *1/*1 | 30 | 9.46 |
| /*2 | 57 | 17.98 |
| *1/*3 *2/*4 | 72 | 22.71 |
| *1/*4 | 6 | 1.89 |
| *1/*6 | 1 | 0.32 |
| *1/*7 *2/*6 | 7 | 2.21 |
| *1/*8 *3/*6 *4/*7 | 1 | 0.32 |
| *2/*2 | 39 | 12.30 |
| *2/*3 | 55 | 17.35 |
| *2/*7 | 3 | 0.95 |
| *2/*8 *3/*7 | 1 | 0.32 |
| *2/*9 | 1 | 0.32 |
| *3/*3 | 35 | 11.04 |
| *3/*7 | 2 | 0.63 |
| *4/*4 | 5 | 1.58 |
| *5/*5 | 1 | 0.32 |
| *5/*9 | 1 | 0.32 |
| Low activity genotypes^{c} | 42 | 13.26 |
| Intermediate activity | 138 | 43.54 |
| genotypes^{d} | | |

^{a}In bold: Genotypes considered to evaluate allelic frequencies.
^{b}167/317 Caucasian; 150/317 African-American subjects.
^{c}With two low activity alleles.
^{d}With one low activity allele.

### EXAMPLE VII

### Relative Expression of the UGT1A7 and UGT1A9 Variants and SN-38 Glucuronidation Activities of UGT1A7 and UGT1A9 Allozymes

Material and Methods

UGT1A7 and UGT1A9 Expression Studies

All five novel UGT1A7 variant alleles were generated by PCR site-directed mutagenesis using pcDNA3-vector containing either UGT1A7*1, *2, *3 or *4 variant alleles as the starting construction. Primers having SEQ ID NO: 32, 33, 34 and 35 (Table 7) were used for site-directed mutagenesis. The variant alleles *5 (SEQ ID NO: 50) and *6 (SEQ ID NO: 51) were generated using *1 (SEQ ID NO: 46) as the template, the *7 (SEQ ID NO: 52) and *9 (SEQ ID NO: 54) variants were obtained using the *2 (SEQ ID NO: 47) allele as template and the *8 (SEQ ID NO: 53) was created from *3 (SEQ ID NO: 48) allele. Expression constructs for the UGT1A9 cDNA sequence construct and constructs for the two nonsynonymous cSNPs were created using the same strategy; The expression plasmid pcDNA3-UGT1A9*1 was obtained by subcloning the Bam HI-Xho I fragment of pBK-CMV/UGT1A9*1 (kindly provided by Dr Alain Belanger from CHUL Research Center, Laval University, Quebec, Canada) into the Bam HI-Xho I site of pcDNA3 expression vector. Mutations were all verified by sequencing. Stable HEK293 cells were transfected with variant pcDNA3-UGT1A7 and pcDNA3-UGT1A9 expression plasmids using the following procedure that has been described previously (Guillemette et al., 2000, Pharmacogenetics, 10: 629-640). HEK293 cells in the exponential growth phase were seeded at a density of 3.25×10⁶ cells/culture dish. Briefly, cells were grown in Dulbecco's-modified Eagle's medium (DMEM) containing 10% fetal bovine serum (FBS), 1% Sodium Pyruvate (NaPy) and 0.1 mg/mL Amikacin in a humidified incubator at 37° C. with an atmosphere of 5% CO₂. The next day, cells at 60% of confluence were washed with DMEM without FBS. Then, cells were incubated with 5 mL of the same medium containing 30 μL Exgen 500™ (MBI fermentas, Burlington, ON, Canada) and 15 μg of the appropriate pcDNA3-UGT expression plasmids. Transfections were stopped after 3 hours by the addition of fresh DMEM with 10% FBS. After 48 hours, geneticin (1 mg/mL) (Invitrogen life technologies, Carlsbad, Calif.) was added to begin the selection process. During the following 4 weeks, fresh medium with antibiotic was added every 2 days until colonies of resistant cells became visible and for amplification of geneticin-resistant cell populations.

Microsomes were prepared by differential centrifugation. The crude cell extracts were centrifuged at 12000×g at 4° C. for 22 min to remove nuclei and other cellular debris. Supernatants were centrifuged at 105000×g for 60 min at 4° C. to obtain the membrane fraction, which was homogenized in the buffer described above. Protein concentrations were determined using the Bradford method according to the manufacturer's recommendations.

To determine the level of UGT proteins expressed in the microsomal fractions obtained from the stably transfected cells, Western blot analyses were conducted as follows. Microsomal proteins (10 μg) from HEK293 cells stably expressing human UGT1A9 and UGT1A7 variants were separated by 10% SDS-polyacrylamide gel electrophoresis. The separated proteins were transferred onto nitrocellulose membranes and probed with the antihuman UGT1A antiserum RC71 (1:1000 dilution) specific for the conserved C-terminal region of the protein. In order to normalize sample loading, blots were re-probed with anti-calnexin antibody (1:2000 dilution; StressGen Biotechnologies Corp., Victoria, Canada), to detect a second ER-resident protein. A donkey antirabbit IgG antibody conjugated with the horseradish peroxidase (Amersham Corp., Oakville, Canada) was used as the secondary antibody (1:10000 dilution). The resulting immunocomplexes were visualized using an enhanced chemiluminescence kit (ECL) (Renaissance, Quebec, Canada) and exposed on Kodak™ XB-1 film. The relative levels of UGT1A allozymes and calnexin were determined by integrated optical density (IOD) using Bioimage programs visage 110S (Genomic solution Inc., Ann Arbor, Mich., USA) and compared to the *1 respective UGT1A9 (SEQ ID NO: 36) and UGT1A7 (SEQ ID NO. 60) alleles.

Western blot analyses of UGT1A7 and UGT1A9 variants expressed in HEK293 cells were performed on microsomal proteins (10 μg) separated on a 10% SDS-polyacrylamide gel. After transferring the proteins, the membranes were probed with an anti-UGT1A RC-71 polyclonal antibody and with an anti-calnexin antibody. The relative levels of UGT1A9 (a) and UGT1A7 proteins (b) were determined by semi-quantitative densitometric analysis of the Enhanced chemiluminescence (ECL) image. The in vitro SN-38 activity was assessed using microsomal fractions prepared from HEK293 cells expressing the *1 and variant UGT1A9 (c) and UGT1A7 (d) alleles and incubated with 5 μM of SN-38 as described in Materials and Methods.

Results

Recombinant Allozyme Western blot Analysis.

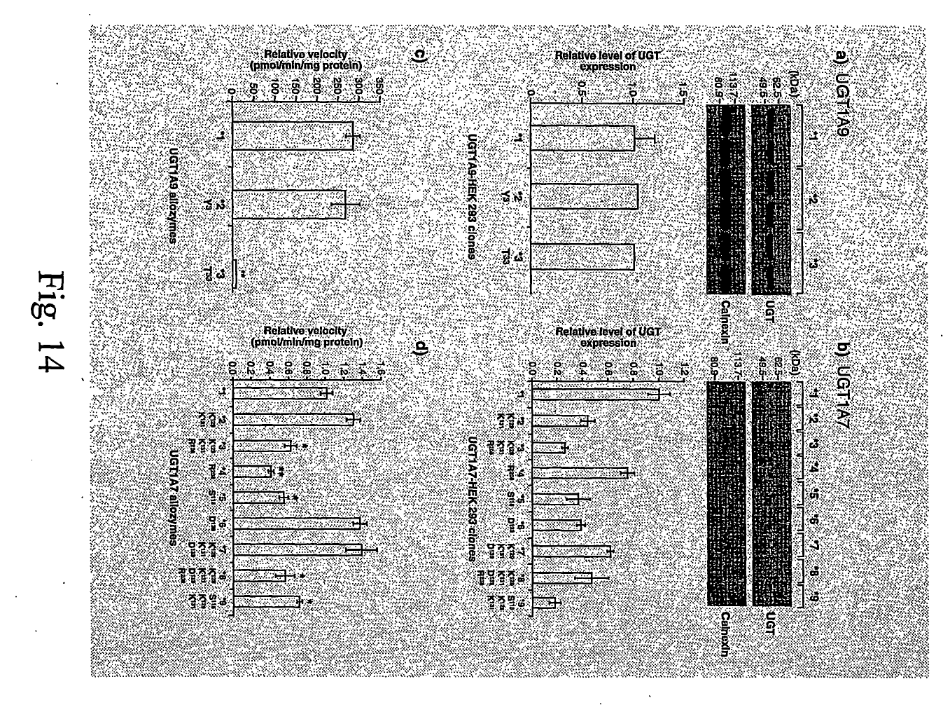

Semi-quantitative Western blot analyses (FIGS. 14a and 14b) showed high levels of immunoreactive UGT protein In all membrane fractions from HEK293 cell lines stably expressing UGTs. An anti-calnexin polyclonal antibody was also used in combination as an internal reference. Significant expression of all UGT1A7 and UGT1A9 alleles was found adequate allowing enzymatic assays to be performed.

### EXAMPLE VIII

### Loss of Function Variants of the UGT1A7 and UGT1A9 Enzymes

Material and Methods

Enzyme Assays

Recombinant allozymes were assayed for UGT activity with the two anticancer agents, SN-38 and flavopiridol, as substrates. Microsomal fractions from HEK293 (40 to 60 μg) were added to a reaction mixture (100 μL) containing 50 mM Tris-HCl, pH 7.3, 10 mM MgCl₂, 100 μg/mL phosphatidylcholine and 2 mM UDP-glucuronic acid. SN-38 was added in concentrations ranging from 0.1 to 200 μM whereas flavopiridol was used at two concentrations: 5 and 200 μM. Commercially available human liver microsomes (Human Cell Culture Center Inc., Laurel, Md.) were incubated in the same conditions for all experiments. Time-course experiments were performed to determine the linearity of the glucuronidation reaction. For the determination of Vₘₐₓ and Kₘ. HEK293 cells stably expressing UGT1A9 enzymes were incubated in the presence of various concentrations of SN-38 ranging from 0.1 to 200 μM and incubated for 30 min as described above whereas UGT1A7 membranes preparations were incubated for 3 hours. All reaction rates were shown to be linear in these conditions. Reactions with SN-38 were stopped by the addition of 200 μL MeOH+1% HCl 2N, followed by centrifugation at 14000×g for 10 minutes. The supernatants were filtered through a 0.22 μm membrane and 100 μL of water was added to the filtrate. For the detection of SN-38 and its glucuronide (SN-38G), 10 μL samples were injected in a liquid chromatographic system (HPLC) coupled to fluorescence detector as described below.

A HPLC method was developed to quantify the rates of SN-38 glucuronidation from the various microsomal fractions under study. The HPLC system used was an Alliance 2695 (Waters, Milford, Mass.) equipped with a 50×3.2 mm Colombus C18 column (Phenomenex, Torrance, Calif.). The chromatographic separation was achieved with a two-solvent gradient system: solvent A (water+1 mM ammonium formate); solvent B (MeOH+1 mM ammonium formate). A linear gradient starting at 20% solvent B was generated over a 3 min period and at a constant flow rate (0.7 mL/min) until a plateau was reached at 65% solvent B and held for 0.8 min. Then a second gradient ranging from 65% to 95% solvent B was generated during the following 2 min. Finally, the column was re-equilibrated to 20% solvent B for 2 min. The column was connected to a fluorescence detector model 474 (Waters, Milford, Mass.) and the molecules were excited at a wavelength of 370 nm and an emission of 425 nm. The retention times for SN-38 and SN-38G were 4.49 and 3.12 min, respectively. Because we could not perform kinetic analysis with the UGT1A9*3 using the previously used electrospray ion-trap mass spectrometry method, the fluorescence detection was preferred since it was more sensitive in these conditions and allowed the detection of SN-38G formed by UGT1A9*3 microsomes at low concentrations of SN-38. Kₘ calculated for the human liver microsomes using both analytical methods were shown to be similar (6.8±3.0 μM with the LCQ detector and 4.8±0.8 μM with the fluorescent detection (data not shown)). Glucuronidation assay using flavopiridol as substrate were performed as previously described (Ramirez et al., 2002, Pharm. Res. 19: 588-594). Relative glucuronidation activities for flavopiridol (5 and 7 glucuronides) were determined for one hour using 5 and 200 μM of substrate and in the same experimental conditions as used for SN-38.

Results

Recombinant UGT1A7 and UGT1A9 Enzyme SN-38 Kinetics.

The functional genomic studies were focused on two anticancer drugs, SN-38 and Flavopiridol. UGT1A7 was previously shown to have the highest intrinsic clearance with SN-38 as substrate along with UGT1A1 and UGT1A9 (Gagne et al., 2002, Mol. Pharmacol. 62:608-617) whereas UGT1A9 is the main UGT involved in the metabolism of flavopiridol (Ramirez et al., 2002, Pharm. Res. 19: 588-594).

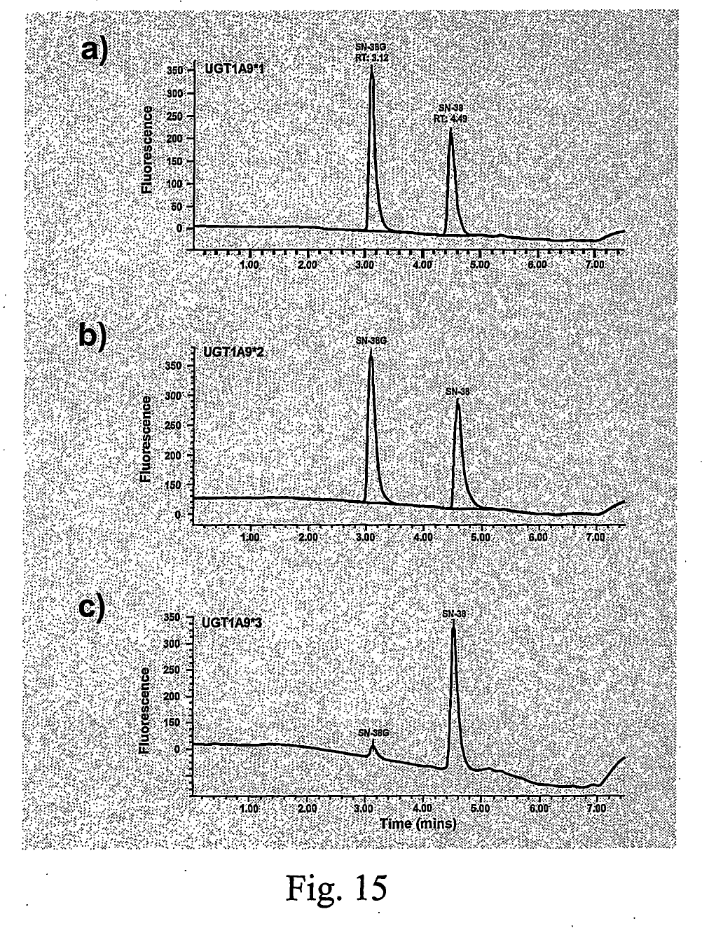

The chromatograms obtained after separation of the reaction products following enzymatic assays with 5 μM of SN-38 and the UGT1A9 variant allozyme preparations are depicted in FIG. 15a), b) and c) *3. The formation of SN-38G by the UGT1A9*3 enzyme is markedly reduced, with only 3.8% residual activity compared to the wild type enzyme (FIG. 15c). Our results thus demonstrate that the M³³T polymorphism dramatically impairs the conjugation rate of SN-38 whereas no significant effect was observed with the UGT1A9*2 allozyme. In contrast, the formation of flavopiridol-G was not statistically different for UGT1A9*2 and UGT1A9*3 compared to the UGT1A9*1 allele at both low and high concentrations (5 μM and 200 μM of flavopiridol), suggesting a substrate specific impact of this amino acid variation in the UGT1A9 protein.

To determine if the amino acid change at codon 33 affects enzyme activity by an alteration of kinetic properties, glucuronidating activity of UGT1A9 allozymes was assessed using a wide range of SN-38 concentrations (0.1 to 200 μM). A non significant higher apparent Kₘ value for the UGT1A9*2 variants was observed as determined at least in three independent experiments. Both UGT1A9*1 and UGT1A9*3 alleles demonstrated a similar apparent Kₘ of 3.03±0.51 and 3.21±0.95, respectively (Table 9). As a result, decreases in level of enzyme activity observed for the UGT1A9*3 allele could not be attributed to the alterations of substrate affinity. However Vₘₐₓ values were about 26 times lower for UGT1A9*3 compared with UGT1A9*1 (11.89±2.61 versus 316.34±52.03 pmol/min/mg of protein, p<0.002). in the analysis of UGT1A7 allozymes, the highest SN-38 glucuronidating activity was observed for UGT1A7*1, *2, *6 and *9. Three novel low activity alleles were identified and the *5, *7 and *8 alleles presented 38-76% lower rates of SN-38G formation compared to UGT1A7*1, similar to the range of activity of the *3 and *4 alleles previously identified as low SN-38 glucuronidating activity alleles (Gagne et al., 2002, Mol. Pharmacol. 62:608-617).

TABLE 10
Kinetic parameters for SN-38 glucuronidation by human UGT1A9
allozymes

| | | | Catalytic |
|---|---|---|---|
| | | | efficiencies |
| UGT1A9 | Apparent | Vₘₐₓ | Vₘₐₓ/Kₘ |
| allozymes | Kₘ (μM) | (pmol/min/mg protein) | (μL/h/mg) |
| UGT1A9*1 | 3.02 ± 0.51 | 316.34 ± 52.03 | 105 |
| UGT1A9*2 | 5.15 ± 1.81 | 324.38 ± 95.09 | 63 |
| UGT1A9*3 | 3.21 ± 0.95 | 11.89 ± 2.61* | 4 |

The values of apparent Kₘ and Vₘₐₓ for the formation of SN38 glucuronide were determined using microsomal preparations from UGT1A9-HEK293 cells. Values were expressed as the mean±SD of at least three independent experiments performed in duplicate from Lineweaver-Burk plots. p<0.002 compared to UGT1A9*1.

### EXAMPLE IX

### Immunofluorescence Localization of UGT1A9*1, UGT1A9*2 and UGT1A5-3 Proteins

Material and Methods

Immunofluorescence Visualization

One cSNP found in the UGT1A9 first exon was located in the signal peptide, thus immunofluorescence experiments were designed to localize the expressed protein within the cells. Stable HEK293 cells expressing human UGT1A9*1. UGT1A9*2 and UGT1A9*3 and also with cells transfected with pcDNA3 vector alone were seeded on culture slides (VWR Scientific, West Chester, Pa.) and allowed to grow for 18 h. Then, cells were washed three times with PBS and fixed for 20 min with paraformaldehyde 2% (w/v, Sigma, St. Louis, Mo.) in PBS. The slides were washed three times with PBS before permeabilization of the membranes for 40 min in PBS containing Saponin 0.2% (w/v, Sigma, St. Louis, Mo.). After three washes with PBS, the cells were incubated for 30 min with gelatin 0.2% in PBS (w/v, Sigma, St. Louis, Mo.). The permeabilized cells were incubated with a rabbit anti-UGT1A primary antibody (RC-71) at a 1:1000 dilution (v/v) in PBS containing Saponin 0.1% and bovine serum albumin 1.5%. Slides were incubated for 1 h and then washed three times with PBS. A goat anti-rabbit secondary antibody (Alexa Fluor 488, Molecular Probes Inc., Eugene, Oreg.) was added at a 1:400 dilution In the same buffer as the primary antibody, and slides were incubated for 30 min at room temperature In the dark. Cells were then washed three times with PBS. Cell counterstaining was achieved by incubating the slides for 30 sec in the dark at room temperature with a 1:1000 (v/v) dilution of diamidino-2-phenylindole (DAPI, Molecular Probes Inc., Eugene, Oreg.). Finally, cells were washed with PBS and mounted with a mounting medium (Sigma, St. Louis, Mo.). For visualization, a Fluoview confocal microscope (BX-61, Olympus, Melville, N.Y.) with a 100× oil objective was used.

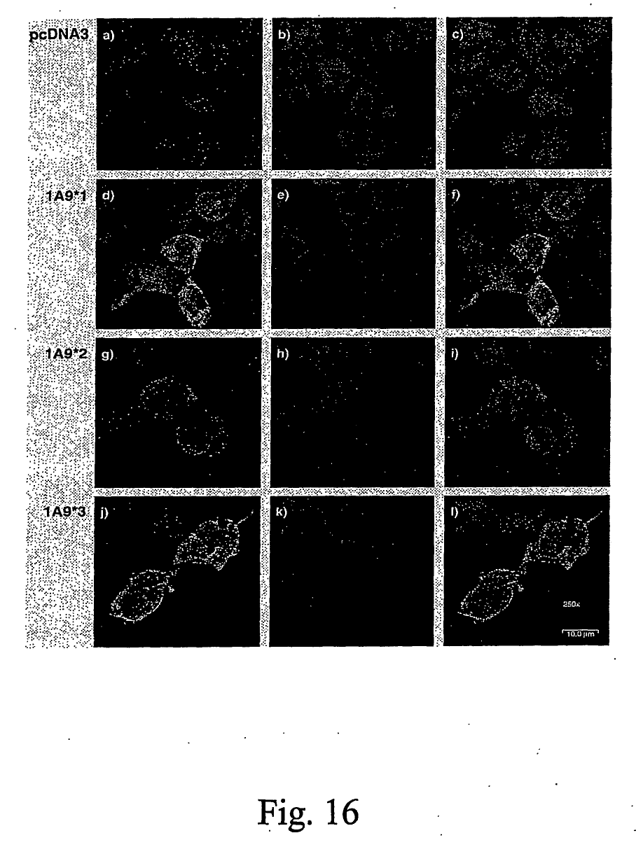

On FIG. 16, HEK293 cells stably expressing pcDNA3 (a) or human UGT1A9 alleles (d), (g), (j) were fixed, permeabilized and then treated with a rabbit anti-UGT1A primary antibody (RC-71), followed by a goat anti-rabbit secondary antibody. Cell counterstaining of the nuclei was performed using DAPI (b), (e), (h), (k). To confirm the localization of the UGT proteins, a combination of the images obtained with the antibodies and the counterstain are shown in (c), (f), (i), (j).

Results

To determine if the subcellular localization of UGT1A9 was affected by the codon 3 mutational polymorphism In the signal peptide region, immunofluorescence experiments were carried out Coloration with diamidino-2-phenylindole (DAPI) was restricted to the nucleus (FIGS. 16e, h and k) whereas the low background observed In the pcDNA3 control vector is due to autofluorescence (FIGS. 16a, b and c). UGT1A9*1, UGT1A9*2 and UGT1A9*3 proteins were localized in the cytoplasm and the perinuclear zone as well as in the endoplasmic reticulum (FIGS. 16d, g and j).

### EXAMPLE X

### Effect of UGT1A1 TATA Box Variations on UGT1A1 Protein Expression and Glucuronidation Activity

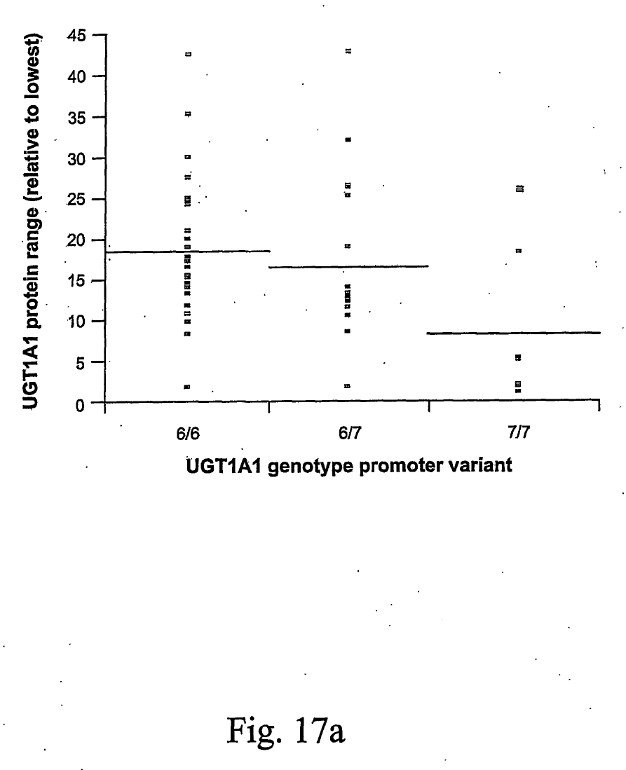

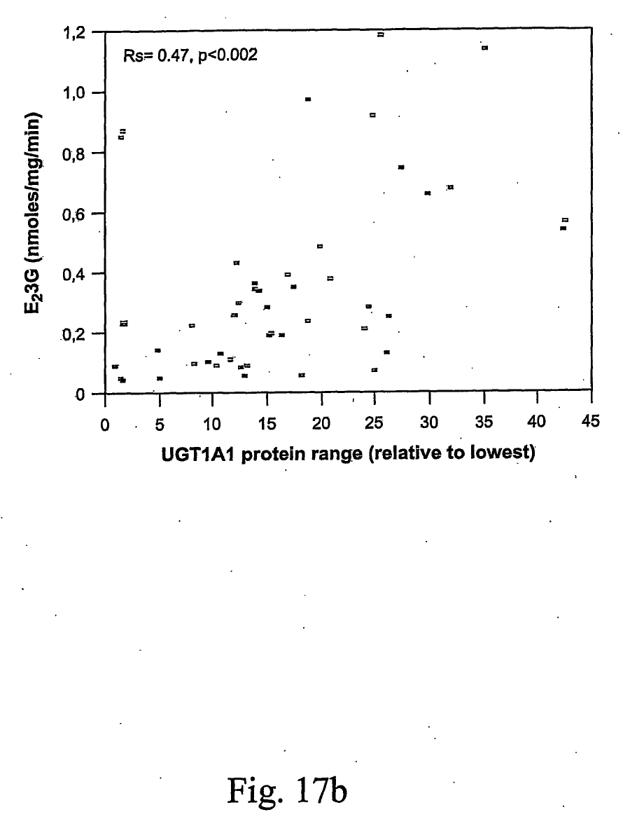

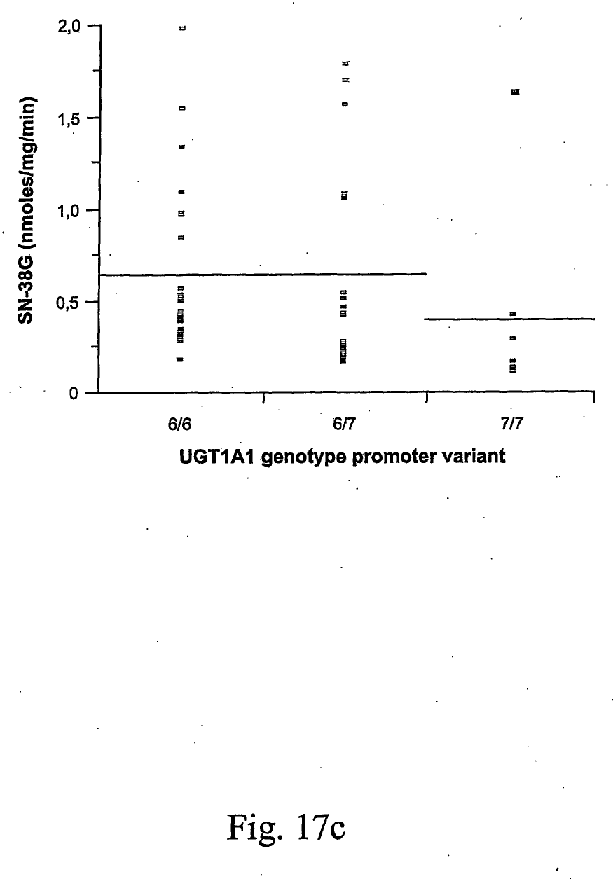

Although a correlative association between TATA box polymorphic variation is reported in prior art, the UGT1A7 and UGT1A9 interindividual variations of the present invention remained unknown at this time and their effect on SN-38 glucuronidation therefore remained unconsidered. In an attempt to decipher Fe particular function of every participating isoform in SN-38-G formation, we were interested to determine whether or not a correlative association could be made between the number of TA repeat in the TATA box of UGT1A1 promoter region and UGT1A1 protein expression even thought novel polymorphic variations were taken into account. As shown in FIG. 17, the presence of TA₆ genotype on both alleles is associated with a higher protein expression while the presence of a TA₇ repeat on only one allele is sufficient to decrease UGT1A1 protein expression. The lowest protein expression level is observed with TA₇ homozygous patients. As shown in FIGS. 17b and 17c, the correlative association is also observed between glucuronidaton of the probe substrate estradiol and the number of TA repeats. A similar correlative association is found with SN-38.

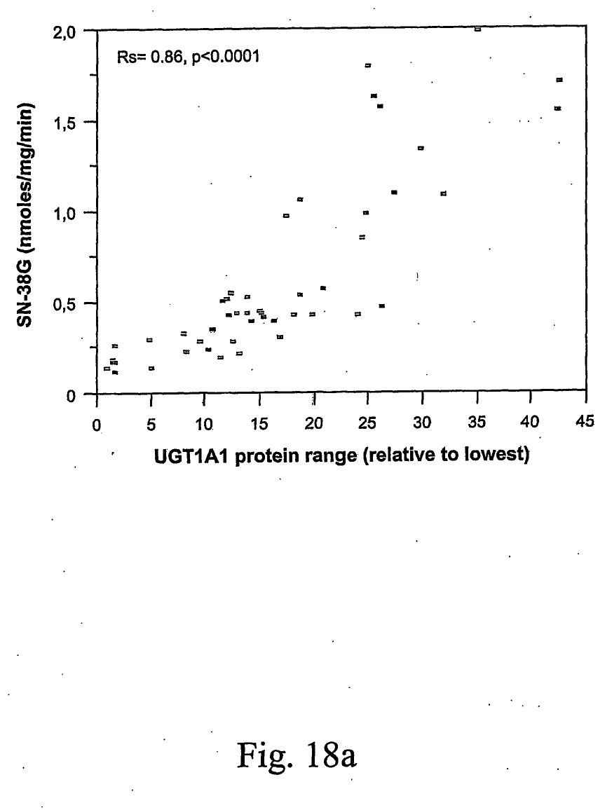

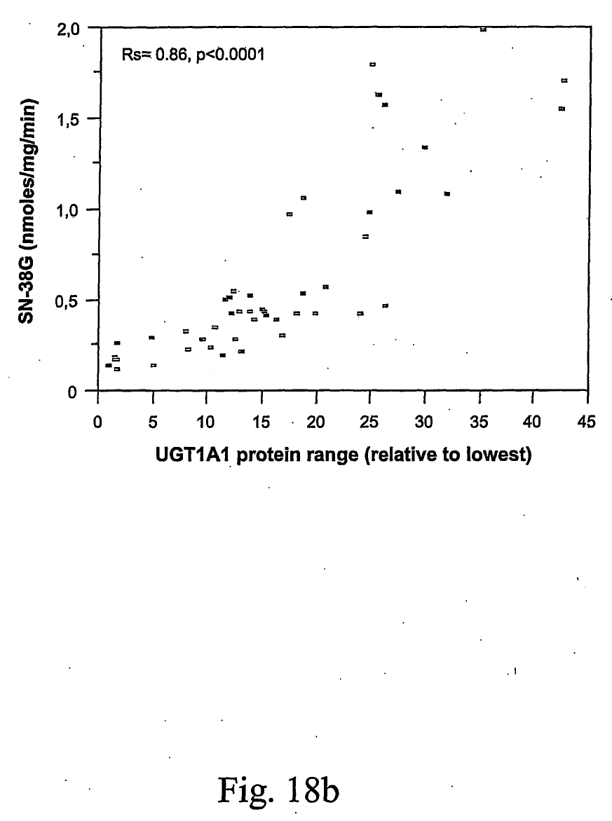

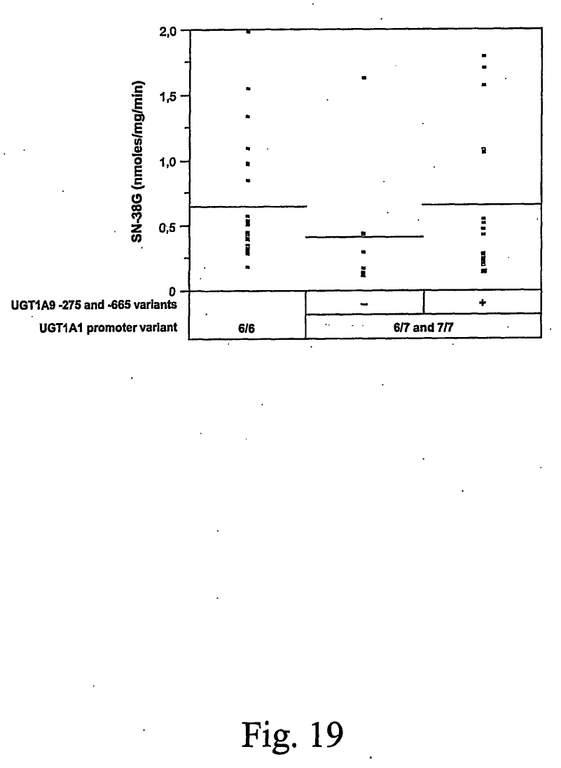

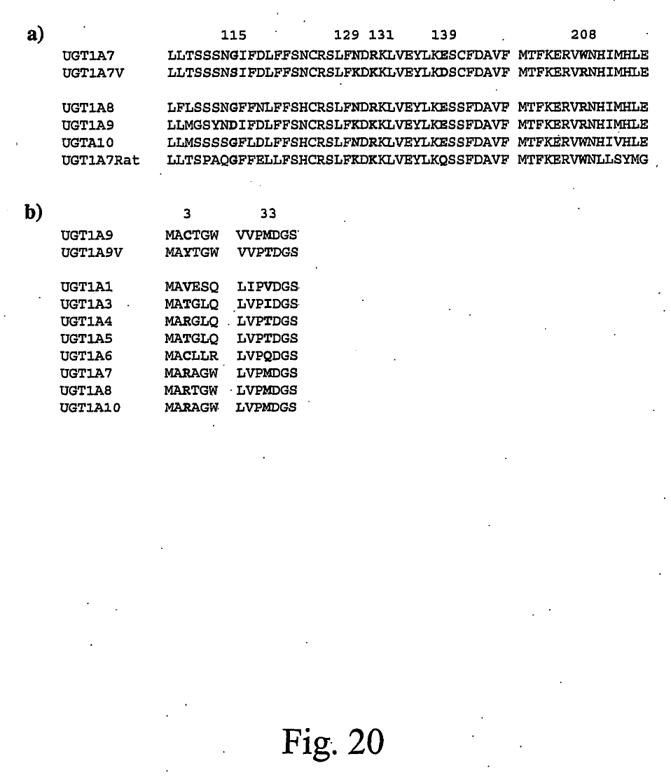

As UGT1A1 is considered as a major SN-38 glucuronidation enzyme, we attempted to determine if an association between the expression of this protein and glucuronide formation could exist As shown in FIG. 18a, there is a positive correlations between glucuronidation of SN-38 and protein level of UGT1A1. To ascertain that the enhancement of glucuronidation observed with this substrate is not attributable to a residual activity of other UGT Isoforms, this experiment was reconducted using probe substrates for UGT1A1, namely estradiol. As seen in FIG. 18b a positive correlation exists between UGT1A1 protein level and estradiol-3-G formation. Since estradiol is an endogenously produced compound and formation of estradiol-3-G is exclusively mediated by UGT1A1, these results demonstrate that a biochemical analysis of serum estradiol-3-G could be properly used to monitor a higher or lower UGT1A1 expression in a patient and therefore, be used as an indicator for determining a predisposition to a physiological reaction to a xenobiotic or an endogenous compound. Finally, FIG. 19 shows the predictive value of the haplotype determination of UGT1A9 and UGT1A1. this haplotype determination includes the genotyping of the UGT1A9 promoter region and the determination of the number of TA repeats in the TATA box of the UGT1A1 promoter, which is a more accurate indicator of SN-38 glucuronidation level than the determination of the TA repeats in the TATA box of the UGT1A1 promoter alone.

### EXAMPLE XI

### Haplotyping the UGT1A Genes

Statistical Analysis

Results were expressed as mean±standard deviation (SD). Differences in kinetic parameters between UGT allelic variants were evaluated for statistical significance by paired Student's t test. All tests were two-sided. The haplotype frequencies will be estimated using the PHASE 1.0.1 software and Hardy-Weinberg equilibrium and linkage disequilibrium analyses will be performed using ARLEQUIN 2.0™ software.

Results

Analysis of the Haplotypic Structure of the UGT1 Gene in Subjects with UGT1A9*1 or UGT1A9*3 alleles.

Haplotypes of the UGT1A gene were analyzed in subjects with the UGT1A9*1/*3 low SN-38 glucuronidation activity genotype.

TABLE 11
UGT1A9 promoter haplotype analysis

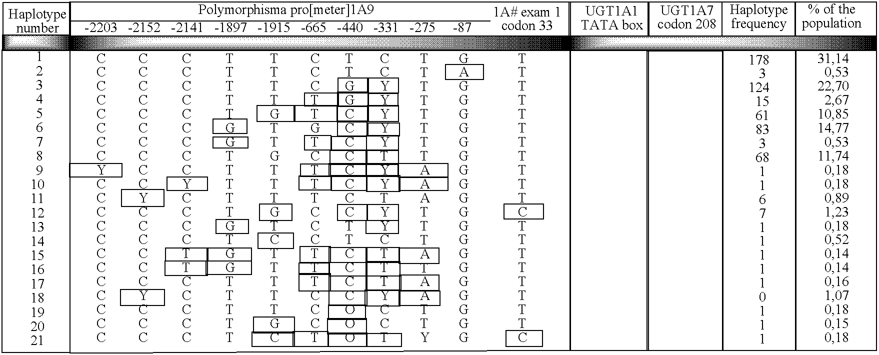

TABLE 12
UGT1A9 and UGT1A1 promoters haplotype analysis

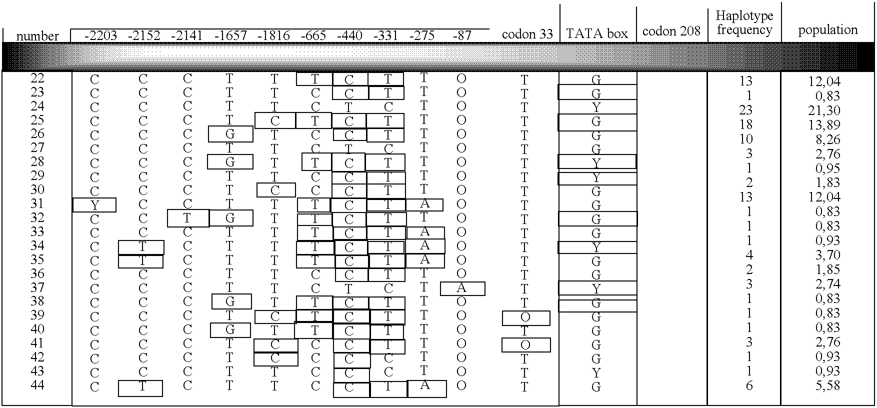

TABLE 13
UGT1A9, UGT1A1 and UGT1A7 haplotype analysis.

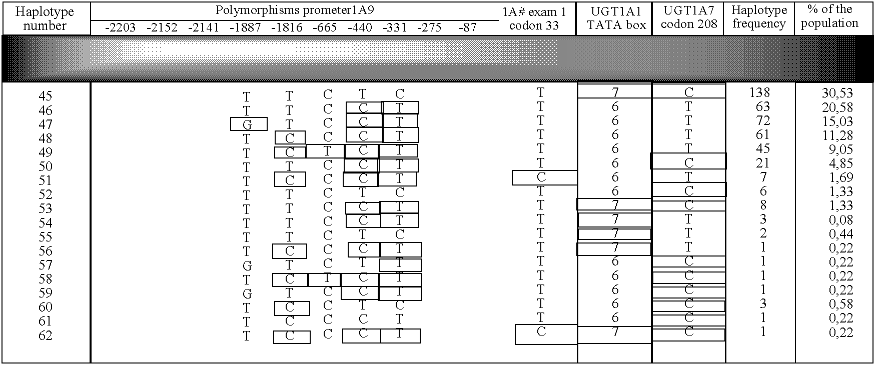

TABLE 14
Allele Frequencies

| | −2208 | | −2152 | | −2141 | | −1887 | | −1818 | |
|---|---|---|---|---|---|---|---|---|---|---|
| | Allele or | Frequency | Allele or | Frequency | Allele or | Frequency | Allele or | Frequency | Allele or | Frequency |
| | genotype | Am | genotype | Am | genotype | Am | genotype | Am | qenotype | Am |
| | C | 0.99 | C | 0.95 | C | 0.99 | T | 0.85 | T | 0.71 |
| | T | 0.01 | T | 0.05 | T | 0.01 | G | 0.15 | C | 0.29 |
| Homo. WT | CC | 0.98 | CC | 0.90 | CC | 0.98 | TT | 0.75 | TT | 0.50 |
| Heterozygous | CT | 0.02 | CT | 0.10 | CT | 0.02 | TG | 0.21 | TC | 0.42 |
| Homo. var. | TT | 0.00 | TT | 0.00 | TT | 0.00 | GG | 0.04 | CC | 0.08 |
| | | N = 48 | | N = 48 | | N = 48 | | N = 48 | | N = 48 |

| | −665 | | −440 | | −331 | | −275 | |
|---|---|---|---|---|---|---|---|---|
| | Allele or | Frequency | Allele or | Frequency | Allele or | Frequency | Allele or | Frequency |
| | genotype | Am | genotype | Am | genotype | Am | genotype | Am |
| | C | 0.58 | T | 0.30 | C | 0.30 | T | 0.92 |
| | T | 0.42 | C | 0.70 | T | 0.70 | A | 0.08 |
| Homo. WT | CC | 0.23 | TT | 0.15 | CC | 0.15 | TT | 0.85 |
| Heterozygous | CT | 0.71 | TC | 0.31 | CT | 0.31 | TA | 0.15 |
| Homo. var. | TT | 0.08 | CC | 0.54 | TT | 0.54 | AA | 0.00 |
| | | N = 48 | | N = 48 | | N = 48 | | N = 48 |

TABLE 15
Functional UGT1A1, UGT1A7 and UGT1A9 SNPs frenquence in
the French-Canadian population.

| | UGT1A9 | | UGT1A7 | | UGT1A1 | |
|---|---|---|---|---|---|---|
| | Codon 33 | | Codon 208 | | TATA box | |
| Wild-type allele | T | 0.98 | T | 0.62 | 6 | 0.67 |
| Mutant allele | C | 0.02 | C | 0.38 | 7 | 0.33 |

### EXAMPLE XII

### Multiple Protein Sequence Alignment of UGT1A Proteins at Selected Positions

UGT1A7*1; UGT1A9*1 and their genetic variant proteins UGT1A7 (a) and UGT1A9 (b) are aligned with close members of the UGT1A subfamily and the rat UGT1A7 isoenzyme. The varying amino acid positions are indicated with bold characters.

Discussion

After resequencing the first exons of UGT1A7 and UGT1A9 genes, 4 polymorphic sites in the targeted regions were identified. Two polymorphic UGT1A9 variants were discovered, UGT1A9* C³Y and UGT1A9*3 M³³T. In addition, the presence of two novel nonsynonymous UGT1A7 SNPs, G¹¹⁵ and E¹³⁹D, combined with previously described missense polymorphisms at codons 129/131 and 208, generated five additional UGT1A7 alleles (*5 through *9). Based on the in vitro functional genomic assays, the UGT1A7*3, *4, *5, *8 and *9 alleles and the UGT1A9*3 allele were all identified as low SN-38 glucuronidating alleles. Results demonstrate that the coinheritance of UGT1A1, UGT1A7 variants and especially the loss of function UGT1A9 polymorphism determine individual's susceptibility to irinotecan-induced toxicity. Thus, findings lay emphasis on the necessity to analyze combination of UGT1A1, UGT1A7 and UGT1A] polymorphisms (haplotypes) rater than looking for a single polymorphism present in the UGT1A1 gene to predict patients at higher risk of developing irinotecan-induced toxicity in a clinical setting.

While the Invention has been described in connection with specific embodiments thereof, it will be understood that it is capable of further modifications and this application is intended to cover any variations, uses, or adaptations of the invention following, in general; the principles of the invention and including such departures from the present disclosure as come within known or customary practice within the art to which the invention pertains and as may be applied to the essential features hereinbefore set forth, and as follows in the scope of the appended claims.

1. A method for determining predisposition to a physiological reaction of an individual to a biologically active compound comprising characterizing nucleotide sequence of at least one of the UGT1A1, UGT1A7 or UGT1A9 gene or a part thereof of said individual, wherein the presence of at least one polymorphic or haplotypic variation in said nucleotide sequence is indicative of said predisposition to a physiological reaction.

2. The method of claim 1, wherein said predisposition is a hereditary predisposition.

3. The method of claim 1, wherein said predisposition is a higher or lower susceptibility, sensibility, diathesis, proneness, proclivity, tendency, sensitivity, responsiveness, resistance or constitutional sickness to said physiological reaction.

4. The method of claim 1, wherein said physiological reaction is a beneficial reaction.

5. The method of claim 1, wherein said physiological reaction is an adverse reaction or a side effect.

6. The method of claim 1, wherein said biologically active compound is a xenobiotic.

7. The method of claim 6, wherein said xenobiotic is a drug, a carcinogen or a pre-carcinogen.

8. The method of claim 7, wherein said drug is an anti-cancer agent or an immunosuppressive agent.

9. The method of claim 8, wherein said anti-cancer agent is a camptothecin or an analog thereof.

10. The method of claim 9, wherein said camptothecin analog is 7-ethyl-10-[4-(1-piperidino)-1-piperidino] carbonyloxy camptothecin (irinotecan, CPT-11), 7-ethyl-10-hydroxycamptothecin (SN-38).

11. The method of claim 8, wherein said immunosuppressive agent is mycophenolic acid (MPA).

12. The method of claim 1, wherein said individual is a human or an animal.

13. The method of claim 1, wherein said individual is a patient with cancer.

14. The method of claim 13, wherein said patient has a colorectal cancer or a solid tumor.

15. The method of claim 1, wherein determining genetic sequence is performed on a DNA or a RNA sample.

16. The method of claim 1, wherein said polymorphic or haplotypic variation is a UGT1A9 variation.

17. The method of claim 16, wherein said UGT1A9 variation is at least one of a C⁻²²⁰⁸T substitution, a C⁻²¹⁵²T substitution, a C⁻²¹⁴¹T substitution, a T⁻¹⁸⁸⁷G substitution, a T⁻¹⁸¹⁸C substitution, a C⁻⁶⁶⁵T substitution, a T⁻⁴⁴⁰C substitution, a C⁻³³¹T substitution, a T²⁷⁵A substitution, a G⁻⁸⁷A substitution, a G⁸A missence mutation (C³Y), a T⁹⁸C missence mutation (M³³T) or combination thereof.

18. The method of claim 17, wherein said G⁸A missence mutation is associated with a decreased predisposition or susceptibility to an anti-cancer agent.

19. The method of claim 17, wherein said G⁸A missence mutation is associated with a decreased responsiveness to an immunosuppressive agent.

20. The method of claim 17, wherein said T⁹⁸C missence mutation is associated with an increased adverse reaction to an anti-cancer agent.

21. The method of claim 1, wherein said polymorphic or haplotypic variation is a UGT1A7 variation.

22. The method of claim 21, wherein said UGT1A7 variation is a G³⁵³T missense mutation, a T³⁹⁷G missense mutation, a C⁴⁰¹A missense mutation, a G⁴⁰²A missense mutations, a G⁴²⁷C missense mutation, a T⁶³²C missense mutation or combination thereof.

23. The method of claim 1, wherein said polymorphic or haplotypic variation is a UGT1A1 variation.

24. The method of claim 23, wherein said UGT1A1 variation is a TA₇ mutation in the TATA box.

25. An isolated nucleotide sequence comprising at least one nucleotide sequence selected from the group consisting of SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, a fragment or the complementary sequences thereof, for determining predisposition to a physiological reaction.

26. The nucleotide sequence of claim 25, wherein said sequence is an allelic variant of UGT1A1, UGT1A7 or UGT1A9.

27. An isolated amino acid sequence comprising at least one amino acid sequence selected from the group consisting of SEQ ID NO: 69, SEQ ID NO: 70, SEQ ID NO: 71 or a fragment thereof.

28. The amino acid sequence of claim 27, wherein said sequence is encoded by a nucleotide sequence comprising at least one sequence selected from the group consisting of SEQ ID NO: 36, SEQ ID NO: 37, SEQ ID NO: 38, a fragment or the complementary sequences thereof.

29. The amino acid sequence of claim 27, wherein the expression of said sequence is regulated by a nucleotide sequence comprising at least one sequence selected from the group consisting of SEQ ID NO: 39, SEQ ID NO: 40, SEQ ID NO: 41, SEQ ID NO: 42, SEQ ID NO: 43, SEQ ID NO: 44, SEQ ID NO: 45 SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, a fragment or the complementary sequences thereof.


SEQUENCE LISTING (71 sequences)

SEQ ID NO: 1 - DNA, 17 bp, Homo sapiens
  primer_bind (1)...(17): UGT1A9 #37 (Forward)
gtgctggtatttctccc

SEQ ID NO: 2 - DNA, 24 bp, Homo sapiens
  primer_bind (1)...(24): UGT1A9 #38 (Reverse)
gtcaaaaatgtcattgtatgaacc

SEQ ID NO: 3 - DNA, 20 bp, Homo sapiens
  primer_bind (1)...(20): UGT1A9 #39 (Forward)
gatctggaccgggagttcaa

SEQ ID NO: 4 - DNA, 22 bp, Homo sapiens
  primer_bind (1)...(22): UGT1A9 #40 (Reverse)
gtgtggctgtagagatcatact

SEQ ID NO: 5 - DNA, 25 bp, Homo sapiens
  primer_bind (1)...(25): UGT1A9 #41 (Forward)
catgcacttggaggaacatttatta

SEQ ID NO: 6 - DNA, 18 bp, Homo sapiens
  primer_bind (1)...(18): UGT1A9 #42 (Reverse)
gagtacacgcattggcac

SEQ ID NO: 7 - DNA, 18 bp, Homo sapiens
  primer_bind (1)...(18): UGT1A7 #18 (Forward)
cgctggacggcaccattg

SEQ ID NO: 8 - DNA, 22 bp, Homo sapiens
  primer_bind (1)...(22): UGT1A7 #17 (Reverse)
gctaaaggggagataacttacc

SEQ ID NO: 9 - DNA, 17 bp, Homo sapiens
  primer_bind (1)...(17): UGT1A7 #122 (Forward)
gctggacggcaccattg

SEQ ID NO: 10 - DNA, 19 bp, Homo sapiens
  primer_bind (1)...(19): UGT1A7 #123 (Reverse)
ccctaagagaagtctgggg

SEQ ID NO: 11 - DNA, 17 bp, Homo sapiens
  primer_bind (1)...(17): UGT1A9 #7 (Forward)
ctcccacctactgtatc

SEQ ID NO: 12 - DNA, 17 bp, Homo sapiens
  primer_bind (1)...(17): UGT1A9 #8 (Forward)
gttcaaggcttttgccc

SEQ ID NO: 13 - DNA, 17 bp, Homo sapiens
  primer_bind (1)...(17): UGT1A9 #9 (Forward)
catttattatgccaccg

SEQ ID NO: 14 - DNA, 16 bp, Homo sapiens
  primer_bind (1)...(16): ASO UGT1A9 C3 (Forward)
atggcttgcacagggt

SEQ ID NO: 15 - DNA, 16 bp, Homo sapiens
  primer_bind (1)...(16): ASO UGT1A9 Y3 (Forward)
atggcttacacagggt

SEQ ID NO: 16 - DNA, 17 bp, Homo sapiens
  primer_bind (1)...(17): ASO UGT1A9 M33 (Forward)
agtgcccatggatggga

SEQ ID NO: 17 - DNA, 17 bp, Homo sapiens
  primer_bind (1)...(19): ASO UGT1A9 T33 (Forward)
agtgcccacggatggga

SEQ ID NO: 18 - DNA, 17 bp, Homo sapiens
  primer_bind (1)...(17): ASO UGT1A7 G115 (Forward)
catccaatggtattttt

SEQ ID NO: 19 - DNA, 17 bp, Homo sapiens
  primer_bind (1)...(17): ASO UGT1A7 S115 (Forward)
catccaatagtattttt

SEQ ID NO: 20 - DNA, 19 bp, Homo sapiens
  primer_bind (1)...(19): Taqman UGT1A7 codon 139/131  #387 (Forward)
gcaccattgcgaagtgcat

SEQ ID NO: 21 - DNA, 22 bp, Homo sapiens
  primer_bind (1)...(22): Taqman UGT1A7 codon 139/131  #388 (Reverse)
ggatcgagaaacactgcatcaa

SEQ ID NO: 22 - DNA, 16 bp, Homo sapiens
  primer_bind (1)...(16): Taqman UGT1A7 codon 139/131  K129/K131-FAM (Forward)
ttaatgaccgaaaatt

SEQ ID NO: 23 - DNA, 17 bp, Homo sapiens
  primer_bind (1)...(17): Taqman UGT1A7 codon 139/131  K129/K131-TET (Forward)
tttaaggacaaaaaatt

SEQ ID NO: 24 - DNA, 26 bp, Homo sapiens
  primer_bind (1)...(26): Taqman UGT1A7 codon 139 #546 (Forward)
gcgaagtgcattttctctattaacaa

SEQ ID NO: 25 - DNA, 20 bp, Homo sapiens
  primer_bind (1)...(20): Taqman UGT1A7 codon 139 #544 (Reverse)
aagccacagcgatcaaaagg

SEQ ID NO: 26 - DNA, 21 bp, Homo sapiens
  primer_bind (1)...(21): Taqman UGT1A7 codon 139 E139-Fam (Forward)
atacttaaaggagagttgttt

SEQ ID NO: 27 - DNA, 21 bp, Homo sapiens
  primer_bind (1)...(21): Taqman UGT1A7 codon 139 D139-Vic (Forward)
atacttaaaggacagttgttt

SEQ ID NO: 28 - DNA, 31 bp, Homo sapiens
  primer_bind (1)...(31): Forward C3Y UGT1A9
gttctctgatggcttacacagggtggaccag

SEQ ID NO: 29 - DNA, 31 bp, Homo sapiens
  primer_bind (1)...(31): Reverse C3Y UGT1A9
ctggtccaccctgtgtaagccatcagagaac

SEQ ID NO: 30 - DNA, 34 bp, Homo sapiens
  primer_bind (1)...(34): Forward M33T UGT1A9
gctactggtagtgcccacggatgggagccactgg

SEQ ID NO: 31 - DNA, 34 bp, Homo sapiens
  primer_bind (1)...(34): Reverse M33T UGT1A9
ccagtggctcccatccgtgggcactaccagtagc

SEQ ID NO: 32 - DNA, 45 bp, Homo sapiens
  primer_bind (1)...(45): Forward E139D UGT1A7
aattagtagaatacttaaaggacagttgttttgatgcagtgtttc

SEQ ID NO: 33 - DNA, 45 bp, Homo sapiens
  primer_bind (1)...(45): Reverse E139D UGT1A7
gaaacactgcatcaaaacaactgtcctttaagtattctactaatt

SEQ ID NO: 34 - DNA, 23 bp, Homo sapiens
  primer_bind (1)...(23): Forward G115S UGT1A7
gttcatccaatagtatttttgac

SEQ ID NO: 35 - DNA, 23 bp, Homo sapiens
  primer_bind (1)...(23): Reverse G115S UGT1A7
gtcaaaaatactattggatgaac

SEQ ID NO: 36 - DNA, 2585 bp, Homo sapiens
  allele (1)...(2585): UGT1A9*1
atggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcagggggcatgaggtggttgtagtcatgccagaggtgagttggcaactgggaagatcactgaattgcacagtgaagacttattcaacttcatataccctggaggatctggaccgggagttcaaggcttttgcccatgctcaatggaaagcacaagtacgaagtatatattctctattaatgggttcatacaatgacatttttgacttatttttttcaaattgcaggagtttgtttaaagacaaaaaattagtagaatacttaaaggagagttcttttgatgcagtgtttctcgatccttttgataactgtggcttaattgttgccaaatatttctccctcccctccgtggtcttcgccaggggaatactttgccactatcttgaagaaggtgcacagtgccctgctcctctttcctatgtccccagaattctcttagggttctcagatgccatgactttcaaggagagagtacggaaccacatcatgcacttggaggaacatttattatgccaccgttttttcaaaaatgccctagaaatagcctctgaaattctccaaacacctgttacggagtatgatctctacagccacacatcaatttggttgttgcgaacggactttgttttggactatcccaaacccgtgatgcccaacatgatcttcattggtggtatcaactgccatcagggaaagccgttgcctatggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctctggtgtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcaattgatccttaaccaataatggtcagtcctcatctctgtcgtgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattctatttaagtgttttcactggtgtcgcatttatttcttgttaagttgcattttctaattacaaaagtaatgcatgattatgacagaaagtttggaaaatatagaggttcacacacacacgccttcattgcgtgtgcatgcataaatgcatgagaaaagaaaaataaccagtaatcacatcgcccagaaataaccccagttacaattgtggcaaatacacatacttataaatattgcagatatattaagtatacc

SEQ ID NO: 37 - DNA, 2585 bp, Homo sapiens
  allele (1)...(2585): UGT1A9*2
atggcttacacagggtggaccagcccccttcctctatgtgtgtgtctgctgctgacctgtggctttgccgaggcagggaagctactggtagtgcccatggatgggagccactggttcaccatgaggtcggtggtggagaaactcattctcagggggcatgaggtggttgtagtcatgccagaggtgagttggcaactgggaagatcactgaattgcacagtgaagacttattcaacttcatataccctggaggatctggaccgggagttcaaggcttttgcccatgctcaatggaaagcacaagtacgaagtatatattctctattaatgggttcatacaatgacatttttgacttatttttttcaaattgcaggagtttgtttaaagacaaaaaattagtagaatacttaaaggagagttcttttgatgcagtgtttctcgatccttttgataactgtggcttaattgttgccaaatatttctccctcccctccgtggtcttcgccaggggaatactttgccactatcttgaagaaggtgcacagtgccctgctcctctttcctatgtccccagaattctcttagggttctcagatgccatgactttcaaggagagagtacggaaccacatcatgcacttggaggaacatttattatgccaccgttttttcaaaaatgccctagaaatagcctctgaaattctccaaacacctgttacggagtatgatctctacagccacacatcaatttggttgttgcgaacggactttgttttggactatcccaaacccgtgatgcccaacatgatcttcattggtggtatcaactgccatcagggaaagccgttgcctatggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctctggtgtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcaattgatccttaaccaataatggtcagtcctcatctctgtcgtgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattctatttaagtgttttcactggtgtcgcatttatttcttgttaagttgcattttctaattacaaaagtaatgcatgattatgacagaaagtttggaaaatatagaggttcacacacacacgccttcattgcgtgtgcatgcataaatgcatgagaaaagaaaaataaccagtaatcacatcgcccagaaataaccccagttacaattgtggcaaatacacatacttataaatattgcagatatattaagtatacc

SEQ ID NO: 38 - DNA, 2585 bp, Homo sapiens
  allele (1)...(2585): UGT1A9*3
atggcttgcacagggtggaccagcccccttcctctatgtgtgtgtctgctgctgacctgtggctttgccgaggcagggaagctactggtagtgcccacggatgggagccactggttcaccatgaggtcggtggtggagaaactcattctcagggggcatgaggtggttgtagtcatgccagaggtgagttggcaactgggaagatcactgaattgcacagtgaagacttattcaacttcatataccctggaggatctggaccgggagttcaaggcttttgcccatgctcaatggaaagcacaagtacgaagtatatattctctattaatgggttcatacaatgacatttttgacttatttttttcaaattgcaggagtttgtttaaagacaaaaaattagtagaatacttaaaggagagttcttttgatgcagtgtttctcgatccttttgataactgtggcttaattgttgccaaatatttctccctcccctccgtggtcttcgccaggggaatactttgccactatcttgaagaaggtgcacagtgccctgctcctctttcctatgtccccagaattctcttagggttctcagatgccatgactttcaaggagagagtacggaaccacatcatgcacttggaggaacatttattatgccaccgttttttcaaaaatgccctagaaatagcctctgaaattctccaaacacctgttacggagtatgatctctacagccacacatcaatttggttgttgcgaacggactttgttttggactatcccaaacccgtgatgcccaacatgatcttcattggtggtatcaactgccatcagggaaagccgttgcctatggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctctggtgtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcaattgatccttaaccaataatggtcagtcctcatctctgtcgtgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattctatttaagtgttttcactggtgtcgcatttatttcttgttaagttgcattttctaattacaaaagtaatgcatgattatgacagaaagtttggaaaatatagaggttcacacacacacgccttcattgcgtgtgcatgcataaatgcatgagaaaagaaaaataaccagtaatcacatcgcccagaaataaccccagttacaattgtggcaaatacacatacttataaatattgcagatatattaagtatacc

SEQ ID NO: 39 - DNA, 2372 bp, Homo sapiens
  allele (1)...(2372): UGT1A9 Haplotype 1
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagtatgagttgccatcttctctggacagagagtatttggttgcctaaaggtaaaatctaaattttgctctgggacaaattccaaaaaaaattagctttaatcaaatttactcttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 40 - DNA, 2372 bp, Homo sapiens
  allele (1)...(2372): UGT1A9 Haplotype 2
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagtatgagttgccatcttctctggacagagagtatttggttgcctaaaggtaaaatctaaattttgctctgggacaaattccaaaaaaaattagctttaatcaaatttactcttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggagtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 41 - DNA, 2372 bp, Homo sapiens
  allele (1)...(2372): UGT1A9 Haplotype 3
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 42 - DNA, 2372 bp, Homo sapiens
  allele (1)...(2372): UGT1A9 Haplotype 4
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 43 - DNA, 2372 bp, Homo sapiens
  allele (1)...(2372): UHT1A9 Haplotype 5
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcacaatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 44 - DNA, 2372 bp, Homo sapiens
  allele (1)...(2372): UGT1A9 Haplotype 6
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccgtaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 45 - DNA, 2372 bp, Homo sapiens
  allele (1)...(2372): UGT1A9 Haplotype 7
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccgtaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 46 - DNA, 2372 bp, Homo sapiens
  allele (1)...(2372): UGT1A9 Haplotype 8
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcacaatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 47 - DNA, 2372 bp, Homo sapiens
  allele (1)...(2372): UGT1A9 Haplotype 9
ctgttttgcctgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgctactaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 48 - DNA, 2372 bp, Homo sapiens
  allele (1)...(2372): UGT1A9 Haplotype 10
ctgttttgcccgggctggagtataatggcgtgatctcagctcaatgcaacctccgcttcccgggttcaagtgattcttctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgctactaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 49 - DNA, 2372 bp, Homo sapiens
  allele (1)...(2372): UGT1A9 Haplotype 11
ctgttttgcccgggctggagtataatggcgtgatctcagctcaatgcaacctccgcttcccgggtttaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgctactaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 50 - DNA, 2372 bp, Homo sapiens
  allele (1)...(2372): UGT1A9 Haplotype 12
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcacaatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccacggatgggagccactggttcaccatgaggtcggtggtggagaaactcattctcaggg

SEQ ID NO: 51 - DNA, 2372 bp, Homo sapiens
  allele (1)...(2372): UGT1A9 Haplotype 13
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccgtaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagtatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 52 - DNA, 2372 bp, Homo sapiens
  allele (1)...(2): UGT1A9 Haplotype 14
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcacaatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagtatgagttgccatcttctctggacagagagtatttggttgcctaaaggtaaaatctaaattttgctctgggacaaattccaaaaaaaattagctttaatcaaatttactcttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 53 - DNA, 2372 bp, Homo sapiens
  allele (1)...(2372): UGT1A9 Haplotype 15
ctgttttgcccgggctggagtataatggcgtgatctcagctcaatgcaacctccgcttcccgggttcaagtgattcttctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccgtaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgctactaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 54 - DNA, 2372 bp, Homo sapiens
  allele (1)...(2372): UGT1A9 Haplotype 16
ctgttttgcccgggctggagtataatggcgtgatctcagctcaatgcaacctccgcttcccgggttcaagtgattcttctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccgtaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 55 - DNA, 2372 bp, Homo sapiens
  allele (1)...(2372): UGT1A9 Haplotype 17
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgctactaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 56 - DNA, 2372 bp, Homo sapiens
  allele (1)...(2372): UGT1A9 Haplotype 18
ctgttttgcccgggctggagtataatggcgtgatctcagctcaatgcaacctccgcttcccgggtttaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgctactaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 57 - DNA, 2372 bp, Homo sapiens
  allele (1)...(2372): UGT1A9 Haploytpe 19
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcataatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttactcttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 58 - DNA, 2372 bp, Homo sapiens
  allele (1)...(2372): UGT1A9 Haplotype 20
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcacaatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtatcttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttactcttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcaggg

SEQ ID NO: 59 - DNA, 2372 bp, Homo sapiens
  allele (1)...(2372): UGT1A9 Haplotype 21
ctgttttgcccgggctggagtataatggcgtgatctcagctcaatgcaacctccgcttcccgggttcaagtgattctcctgcctcagcctccagagtagctgggattacaggcatgcaccaccacctgcagctaattttttgcatttttagtagagatagggtttcaccatgttggccaggctggtctccaactcctggcctcccgtgatacgcccaccttgacctcccaaagtgctgggactacaggtgtgagccaccacgcccaggcacacatagaatttttgactccctaaaaatttactattaatagcctactgtgcactagaagccttaccaataacagaaacagttgcttaacacatatttggcatgttatatgtgttatatactgtattatcacaatgaagtcagctagagaaaagaaaatgttattaagaaaatcttaaggaagagaaaattaagtattcattaagtggaagtggatcatgataaaggtcttcctcttgattgtcctccattgagtaggctgagaaggaggaagaggtgggttggttttgctgtttcaggggtggcagagggggaagaagtggaggaagaaggaggagagacaggtacacttggtgtaactttacagaattacatcataattattatttgacttttttgcctttgcaattctttgaaaatgcttttttacagtactagtccttcttccccatttgctttagtttcagtgcccattcatggaagggtttgtgttgtaaaataagtcaaaagtagtcttaataattggaagcctttgccaaactgtttaataggaatttgttttctggcatggcttcttctatgtcttctttagtatctggtactgattcaaaagcactcatctccatcaagtcatcttctgttgattcctctggtgtggtgtctattcattcttgaatttctcacagattcatatcttgaaagaccatatcccccaccttttgttgctgaattagagatattgggtttgcaggcaagtagaccactttgacaccttcagtgttgaactcatgggttctgggtggctaggggcattgtccaaaaatcaaaagaactttgaaagaccgtctcttactggcaagatattacctgacttcagggacaaagtaatgatagaaccaatccagaaaaagtgttcttgccgaggccttcttgtacaacaaaaaaactggcagtgggtattgatcttttccctttaaggcttggaggctagcaggcttatggatgggggcagtcctatttgtaaacccaaacatacaaacatacaaactatgtcaaaggcatagcatgggtactgtgaaaggagggtgaaaacacaaagttgacatcacctctgacctcaaggagtgctcagcagactgagagagacaagtacatattttcctgaaggagggcactggagtgatggcgtgtttagaatgtgcaagttgagcggtcactgagaggcagctcagcagagtgctctcgcaaggattgggcgggcaacttcccactgcgtgcgatgtattttaggaaagccatttaaaataggagacggttactttccatcaagtccctggtatggtccatggaagcagggttgtcagtctcatttcagcattttagaggcttctcagggtttggaaatggaagaagagaagcagcaatatgtatgcattgcagagacacaggcgagccccaatttaggaggttaggaggtcagtgctaagggccttgttttctttgcttagagcatgagttgccatcttctctggacagagagtatttggttgcctaaaggtaaaatctaaattttgctctgggacaaattccaaaaaaaattagctttaatcaaatttacttttactttatctttctgaaccttcaaggtccaaaagcattggttaataattctgcttctaaacttaacattgcagcacagggcatgttctgcccccaaggcaaagaccataagctactgttgtctggaaaacatacaaatagatatctcagcaaaagctactcatatattcttgttcttttgggtaaatcattgtcagtgactgatttttttttatgaaaggataaaaacacgccctctattggggtcaggttttgtgctggtatttctcccacctactgtatcataggagcttagattcccagctgcttgctctcagctgcagttctctgatggcttgcacagggtggaccagcccccttcctctatgtgtgtgtctgctgctgacctgtggctttgccgaggcagggaagctactggtagtgcccacggatgggagccactggttcaccatgaggtcggtggtggagaaactcattctcaggg

SEQ ID NO: 60 - DNA, 1229 bp, Homo sapiens
  allele (1)...(1229): UGT1A7*1
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaatgaccgaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 61 - DNA, 1229 bp, Homo sapiens
  allele (1)...(1229): UGT1A7*2
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaaggacaaaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 62 - DNA, 1229 bp, Homo sapiens
  allele (1)...(1229): UGT1A7*3
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaaggacaaaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtacggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 63 - DNA, 1229 bp, Homo sapiens
  allele (1)...(1229): UGT1A7*4
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaatgaccgaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtacggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 64 - DNA, 1229 bp, Homo sapiens
  allele (1)...(1229): UGT1A7*5
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatagtatttttgacttatttttttcaaattgcaggagtttgtttaatgaccgaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 65 - DNA, 1229 bp, Homo sapiens
  allele (1)...(1229): UGT1A7*6
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaatgaccgaaaattagtagaatacttaaaggacagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 66 - DNA, 1229 bp, Homo sapiens
  allele (1)...(1229): UGT1A7*7
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaaggacaaaaaattagtagaatacttaaaggacagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 67 - DNA, 1229 bp, Homo sapiens
  allele (1)...(1229): UGT1A7*8
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaaggacaaaaaattagtagaatacttaaaggacagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtacggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 68 - DNA, 1229 bp, Homo sapiens
  allele (1)...(1229): UGT1A7*9
atggctcgtgcagggtggactggcctccttccactatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatagtatttttgacttatttttttcaaattgcaggagtttgtttaaggacaaaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatacaaaaaaaccacagtaagaaatgaaacttccctttttttgctaattctacactacccccagaggaaaatattcttagcagttttgtgtgaattgttttcaatttttttgaaatta

SEQ ID NO: 69 - protein, 530 aa, Homo sapiens
  VARIANT (1)...(530): UGT1A9*1 protein
MACTGWTSPLPLCVCLLLTCGFAEAGKLLVVPMDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 70 - protein, 530 aa, Homo sapiens
  VARIANT (1)...(50): UGT1A9*2 protein
MAYTGWTSPLPLCVCLLLTCGFAEAGKLLVVPMDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 71 - protein, 530 aa, Homo sapiens
  VARIANT (1)...(530): UGT1A9*3 protein
MACTGWTSPLPLCVCLLLTCGFAEAGKLLVVPTDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH